SEQUENCE LISTING (8 sequences)

SEQ ID NO: 1 - protein, 314 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 2 - protein, 314 aa, Homo sapiens
MRIAVICGCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 3 - DNA, 6981 bp, synthetic construct
  source 1..6981: mol_type = other DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatgagaatcgccgtgatctgtttctgtctgctgggcatcacctgtgccattcccgtcaagcaggccgactccggctctagcgaggagaagcagctgtataataaataccccgacgccgtggctacctggctgaaccccgacccaagccagaaacagaacctgctggccccccagaacgccgtgtccagtgaggagaccaatgatttcaagcaggagaccctgccctccaaaagcaacgaaagccacgaccacatggatgacatggatgacgaggacgatgatgatcacgtggactcacaggacagcatcgactccaacgacagcgacgacgtggacgataccgacgattcacaccagtctgacgagagccaccactccgacgagagcgacgaactggtgaccgactttcccacagacctgcctgctaccgaggtgtttaccccagtggtccccactgtggatacctacgacgggcggggcgacagcgtggtctacggcctgcggtccaagtctaaaaaatttaggaggcccgatattcagtaccccgacgccactgacgaagatattacttcacatatggagagcgaggagctgaatggcgcctacaaggccatccccgtggctcaggacctgaacgctcctagcgattgggacagcagaggcaaggatagctacgagaccagtcagctggacgaccagtctgccgagacccattctcacaagcagagccgcctgtacaagcggaaggctaacgacgagagcaacgagcactcagacgtgattgactcccaggagctgagcaaagtgtctcgggagtttcactctcacgaattccactcccacgaggacatgctggtggtggaccccaaaagcaaggaggaggataagcatctgaaattccggatcagtcacgagctggattccgcctccagcgaggtgaacggaagcggagccacgaacttctctctgttaaagcaagcaggagatgttgaagaaaaccccgggcctatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaaacccagctttcttgtacaaagtggtgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggatccctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatctttt

SEQ ID NO: 4 - protein, 15 aa, synthetic construct
GGGGSGGGGSGGGGS

SEQ ID NO: 5 - protein, 6 aa, synthetic construct
GSGSGS

SEQ ID NO: 6 - protein, 5 aa, unidentified
  source 1..5: note = laminin peptide
IKVAV

SEQ ID NO: 7 - protein, 6 aa, synthetic construct
HHHHHH

SEQ ID NO: 8 - protein, 28 aa, Homo sapiens
KQNLLAPQNAVSSEETNDFKQETLPSKS

(19) United States
(12) Patent Application Publication
(10) Pub. No.: US 2026/0356918 A1
(43) Pub. Date: Nov. 12, 2026

(54) OSTEOPONTIN COMPOSITIONS AND METHODS OF USE

(71) Applicant: Biomilq, Inc., Winston-Salem, NC (US)

(72) Inventors: Joydeep Basu, Durham, NC (US); Leila Strickland, Hillsborough, NC (US); Victoria Slaughter, Durham, NC (US); Anne Hucks Moxley, Durham, NC (US); Katherine Richeson, Durham, NC (US)

(21) Appl. No.: 19/489,074

(22) PCT Filed: May 31, 2024
(86) PCT No.: PCT/US2024/032031

§ 371 Date: Dec. 1, 2025

Related U.S. Application Data
(60) Provisional application No. 63/506,017, filed on Jun. 2, 2023.
(60) Provisional application No. 63/563,201, filed on Mar. 8, 2024.

(51) Int. Cl. C07K 14/78; C12N 5/00; C12N 5/071; C12N 15/85

(52) U.S. Cl. CPC C07K 14/78; C12N 5/0062; C12N 5/0631; C12N 15/85; C12N 2500/02; C12N 2510/04; C12N 2513/00

(57) ABSTRACT

Provided herein are recombinant osteopontin compositions and methods of use and production of recombinant osteopontin.

### CROSS-REFERENCE

This application is the U.S. National Stage entry of International Application No. PCT/US2024/032031, filed on May 31, 2024, which claims the benefit of and priority to U.S. Provisional Application No. 63/506,017, filed on Jun. 2, 2023, and U.S. Provisional Application No. 63/563,201, filed on Mar. 8, 2024, the entire disclosure of each of which is incorporated by reference in its entirety herein for all purposes.

### SEQUENCE LISTING

The instant application contains a Sequence Listing which has been submitted electronically in XML format and is hereby incorporated by reference in its entirety. Said XML copy, created on Sep. 10, 2024, is named 00015.007.1201_SL.xml and is 15,698 bytes in size.

### SUMMARY

Disclosed herein, in certain embodiments, are recombinant forms of osteopontin comprising one or more post translational modification sites at positions 33, 36, 42, 66, 73, 237, 258, 267, 270, 291, 308, or 311 of SEQ ID NO: 1 or SEQ ID NO: 2. In some embodiments, the post-translational modification is selected from the group consisting of: ubiquitination, acylation, isoprenylation, prenylation, alkylation, glycosylation, phosphorylation, and a disulfide bridge. In some embodiments, the recombinant osteopontin comprises post translational modification sites at positions 33, 36, 42, 66, 73, 237, 258, 267, 270, 291, 308, and 311. In some embodiments, the recombinant osteopontin is phosphorylated at one or more post translational modification sites at positions 33, 36, 42, 66, 73, 237, 258, 267, 270, 291, 308, or 311 of SEQ ID NO: 1 or SEQ ID NO: 2. In some embodiments, the recombinant osteopontin is glycosylated at one or more post translational modification sites at positions 33, 36, 42, 66, 73, 237, 258, 267, 270, 291, 308, or 311 of SEQ ID NO: 1 or SEQ ID NO: 2. In some embodiments, the recombinant osteopontin is about 35 kilodalton (kDa) in size.

Disclosed herein, in certain embodiments, are methods of producing recombinant osteopontin from mammary cells, the method comprising: (a) culturing a live cell construct in a bioreactor under conditions which produce a cultured milk product, said live cell construct comprising: (i) a three-dimensional scaffold having an exterior surface, an interior surface defining an interior cavity/basal chamber, and a plurality of pores extending from the interior surface to the exterior surface; (ii) a matrix material disposed on the exterior surface of the three-dimensional scaffold; (iii) a culture media disposed within the interior cavity/basal chamber and in fluidic contact with the internal surface; and (iv) an at least 70% confluent monolayer of polarized mammary cells disposed on the matrix material, wherein the mammary cells are modified to overexpress the recombinant osteopontin; and (b) isolating the recombinant osteopontin from the cultured milk product. In some embodiments, the mammary cells are modified to overexpress the recombinant osteopontin by expressing a vector comprising a polynucleotide having a sequence as set forth in SEQ ID NO: 3. In some embodiments, the mammary cells are selected from the group consisting of primary mammary epithelial cells, mammary myoepithelial cells, mammary progenitor cells, immortalized mammary epithelial cells, immortalized mammary myoepithelial cells, and immortalized mammary progenitor cells. In some embodiments, the mammary cells are immortalized mammary epithelial cells. In some embodiments, the mammary cell is human. In some embodiments, the polarized mammary cells comprise an apical surface and a basal surface. In some embodiments, the basal surface of the mammary cells is in fluidic contact with the culture media. In some embodiments, the bioreactor is an enclosed bioreactor. In some embodiments, the bioreactor comprises an apical compartment that is substantially isolated from the internal cavity/basal chamber of the live cell construct. In some embodiments, the apical compartment is in fluidic contact with the apical surface of the mammary cells. In some embodiments, the cultured milk product is secreted from the apical surface of the mammary cells into the apical compartment. In some embodiments, the culture media substantially does not contact the cultured milk product. In some embodiments, total cell density of mammary cells within the bioreactor is at least 10¹¹. In some embodiments, total surface area of mammary cells within the bioreactor is at least 1.5 m². In some embodiments, the culture medium comprises a carbon source, a chemical buffering system, one or more essential amino acids, one or more vitamins and/or cofactors, and one or more inorganic salts. In some embodiments, the matrix material comprises one or more extracellular matrix proteins. In some embodiments, the scaffold comprises a natural polymer, a biocompatible synthetic polymer, a synthetic peptide, a composite derived from any of the preceding, or any combination thereof. In some embodiments, the natural polymer is collagen, chitosan, cellulose, agarose, alginate, gelatin, elastin, heparan sulfate, chondroitin sulfate, keratan sulfate, and/or hyaluronic acid. In some embodiments, the biocompatible synthetic polymer is polysulfone, polyvinylidene fluoride, polyethylene co-vinyl acetate, polyvinyl alcohol, sodium polyacrylate, an acrylate polymer, and/or polyethylene glycol. In some embodiments, the culturing is carried out at a temperature of about 27 °C to about 39 °C. In some embodiments, the culturing is carried out at a temperature of about 30 °C to about 37 °C. In some embodiments, the culturing is carried out at an atmospheric concentration of CO2 of about 4% to about 6%. In some embodiments, the culturing is carried out at an atmospheric concentration of CO2 of about 5%.

Disclosed herein, in certain embodiments, are mammary cells expressing a sequence as set forth in SEQ ID NO: 3. In some embodiments, the mammary cell is selected from the group consisting of: primary mammary epithelial cell, mammary myoepithelial cell, mammary progenitor cell, immortalized mammary epithelial cell, immortalized mammary myoepithelial cell, and immortalized mammary progenitor cell. In some embodiments, the mammary cells is an immortalized mammary epithelial cell. In some embodiments, the mammary cell is human.

These and other aspects of the disclosure are set forth in more detail in the description of the disclosure below.

### BRIEF DESCRIPTION OF THE DRAWINGS

FIG. 1 shows an example of the collection of milk for nutritional use from mammary epithelial cells grown as a confluent monolayer in a compartmentalizing culture apparatus in which either fresh or recycled media is provided to the basal compartment and milk is collected from the apical compartment. TEER, transepithelial electrical resistance.

FIG. 2 shows an example of polarized absorption of nutrients and secretion of milk across a confluent monolayer of mammary epithelial cells anchored to a scaffold at the basal surface.

FIG. 3 depicts an example micropatterned scaffold that provides increased surface area for the compartmentalized absorption of nutrients and secretion of milk by a confluent monolayer of mammary epithelial cells.

FIG. 4 depicts three examples of a hollow fiber bioreactor depicted as a bundle of capillary tubes (top), which can support mammary epithelial cells lining either the external (top and lower left) or internal (lower right) surface of the capillaries, providing directional and compartmentalized absorption of nutrients and secretion of milk.

FIG. 5 depicts a cross-section of three-dimensional cell construct. The construct is made up of a scaffold having an interior surface defining an interior cavity/basal chamber and an exterior surface. The interior cavity/basal chamber comprises cell culture media. A matrix material sits on top of the exterior surface of the scaffold. Pores transverse the scaffold from the interior surface to the exterior surface, allowing cell media to contact the basal surface of the cells of the cell monolayer disposed on the matrix material.

FIG. 6 depicts a bioreactor for producing a cultured milk product. The bioreactor is made up of a cell construct and an apical chamber. The cell construct is made up of a scaffold having an interior surface defining an interior cavity/basal chamber and an exterior surface. The cavity comprises cell culture media. A matrix material sits on top of the exterior surface of the scaffold. Pores transverse the scaffold from the interior surface to the exterior surface, allowing cell media to contact the basal surface of the cells of the cell monolayer disposed on the matrix material. The apical surface of the cells of the cell monolayer secrete the milk/cultured milk product into the apical chamber. The apical chamber and the interior cavity/basal chamber are separated by the cell monolayer.

FIG. 7 depicts an exemplary cell construct. The construct is made up of a scaffold having an interior surface defining an interior cavity/basal chamber and an exterior surface. The interior cavity/basal chamber comprises cell.

FIG. 8 depicts an exemplary cell construct having mammary epithelial cells (MECs) and plasma cells. The plasma cells are adjacent to the scaffold. The MECs form a confluent monolayer above (and in some instances, in between) the plasma cells, with the apical side of the MECs facing the apical compartment (or, milk compartment). The plasma cells secrete IgA, which then binds to a receptor on the basolateral surface of the MECs, triggering internalization of the antibody-receptor complex and further processing of the antibody into sIgA as it transits toward the apical surface (not shown).

FIGS. 9A-9B depict expression of endogenous osteopontin (OPN) in human mammary epithelial cells (hMEC) cultured using a hollow fiber bioreactor. FIG. 9A. shows average production of OPN over 4 days as measured by ELISA. Dots represent individual bioreactors and black line indicates the OPN level in the media input (blank media) used to culture the cells. FIG. 9B. shows Western blot analysis of OPN or recombinant OPN (rOPN) protein from samples of breast milk, infant formula, HEK293 cells expressing human OPN, NSO cells expressing human OPN, Bovine milk-derived OPN, endogenous OPN generated from BMQ1127 cells cultured using hollow fiber bioreactor (HFBR), endogenous OPN generated from BMQ1127 cells treated with prolactin and cultured using hollow fiber bioreactor (HFBR), and endogenous OPN generated from BMQ1036 cells cultured by HFBR.

FIGS. 10A-10C depict overexpression of OPN in immortalized hMEC lines. FIG. 10A shows a schematic of lentiviral vector systems used to overexpress human OPN in immortalized hMEC lines. FIG. 10B shows OPN ELISA analysis of conditioned media from the BMQ1036 Tpb-hMEC line cultured in 2D adherent format for 7 days. FIG. 10C shows the Western blot analysis of OPN from conditioned media across 3 replicates cultured in 2D adherent format for 7 days from two distinct immortalized hMEC lines compared to endogenous levels (WT).

FIGS. 11A-11B show expression of OPN from HEK-293 cells overexpressing OPN in suspension. FIG. 11A shows an exemplary set-up of STR and matched shake flask runs for HEK-293 suspension lines. FIG. 11B shows overall OPN production tracked via ELISA per mL of conditioned media over time. OPN1/OPN2 are over-expressing HEK lines. NORM1/NORM2 are wild-type HEK lines.

FIGS. 12A-12C show expression of OPN from hMECs overexpressing OPN in a hollow fiber bioreactor. FIG. 12A shows an exemplary set-up of a hollow fiber bioreactor. FIG. 12B shows the log scaled average of OPN concentration tracked via ELISA per mg of glucose consumed plotted over time for hMECs. Dots represent values for biological replicate bioreactors. FIG. 12C shows the average OPN concentration tracked via ELISA per mg of glucose consumed for hMECs on day 14. Dots represent values for biological replicate bioreactors. Error bars represent standard deviation between the 4-8 replicate bioreactors.

FIGS. 13A-13C. show phosphoproteomics of OPN in breast milk and HFBR product FIG. 13A shows preliminary phosphorylation sites detected in breast milk pooled from 10 donors determined via untargeted proteomics on phospho-enriched peptides. Yellow indicates unique peptides identified and green phosphorylation sites identified. FIG. 13A discloses SEQ ID NO: 1. FIG. 13B. shows the same as FIG. 13A, except sample is a pooled product from HFBR production run using BMQ 1127 and BMQ 1036 cell lines expressing endogenous OPN. FIG. 13B discloses SEQ ID NO: 1. FIG. 13C shows a comparison of phosphorylation sites identified in FIGS. 13A and 13B. FIG. 13C discloses SEQ ID NO: 2.

FIGS. 14A-14D show phosphoproteomic data of one representative phospho-enriched phosphopeptide of OPN from human donor breast milk (FIG. 14A), commercially available human OPN produced and isolated from HEK293 cells (FIG. 14B), commercially available human OPN produced and isolated from bacterial cells (FIG. 14C), and OPN in conditioned media secreted by OPN-expressing cells isolated from said human donor breast milk and grown in HFBR for 2 weeks (FIG. 14D). FIG. 14A discloses SEQ ID NOs: 8 and 8, respectively, in order of appearance.

FIGS. 15A-15B show an adhesion assay for measuring OPN activity. Conditioned media from HEK-293 suspension cells over-expressing OPN was compared to isolated and commercially available OPN. FIG. 15A shows an exemplary schematic representing a HEK-293 adhesion activity assay for measuring OPN activity. FIG. 15B shows the normalized percentage of HEK-293 cell adhesion plotted as a function of OPN concentration.

FIGS. 16A-16C show suspension adaptation of adherent hMEC. FIG. 16A shows an exemplary schematic of suspension adapting hMEC in spinner flasks. FIG. 16B shows Western blot analysis of conditioned media samples from spinner flasks collected over time for wild type (WT) vs OPN overexpressing (EO) hMEC lines cultured in suspension. FIG. 16C shows the number of viable adherent hMEC measured over time during suspension adaptions.

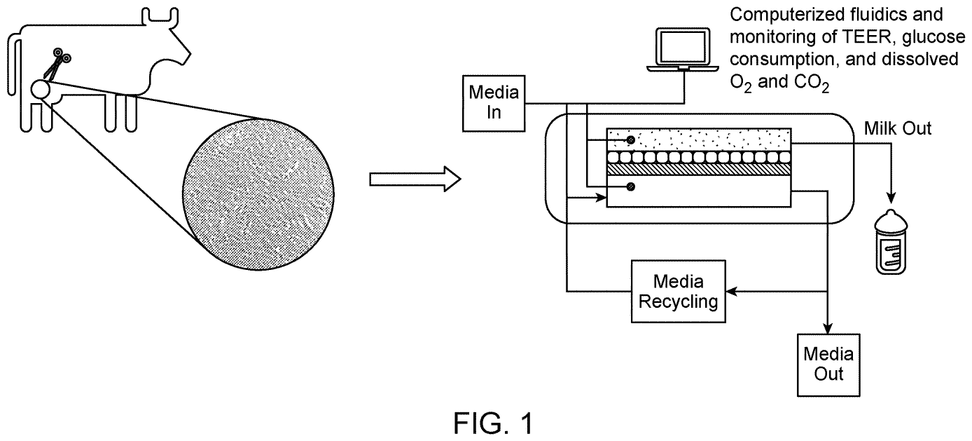

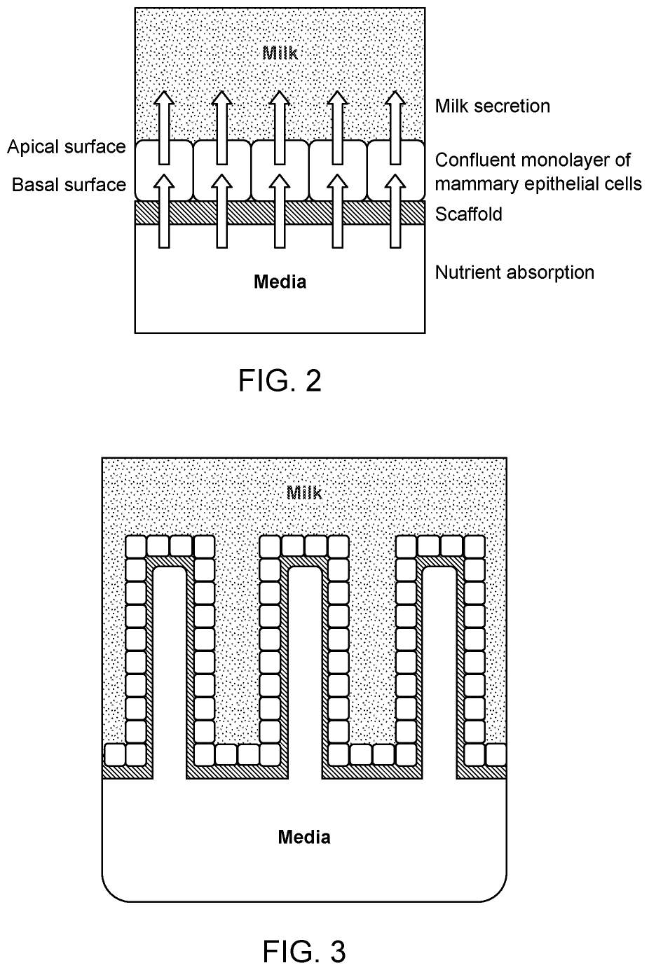

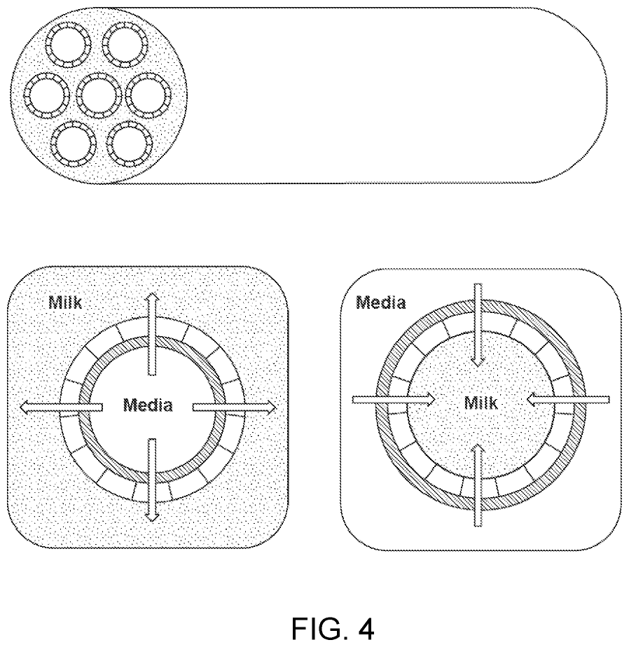

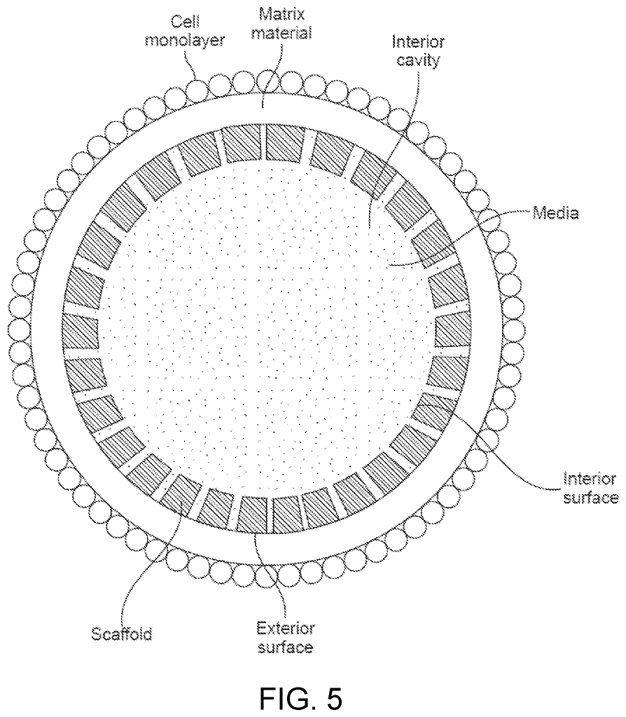

### DETAILED DESCRIPTION

This disclosure is not intended to be a detailed catalog of all the different ways in which the disclosure may be implemented, or all the features that may be added to the instant disclosure. For example, features illustrated with respect to one embodiment may be incorporated into other embodiments, and features illustrated with respect to a particular embodiment may be deleted from that embodiment. Numerous variations and additions to the various embodiments suggested herein will be apparent to those skilled in the art in light of the instant disclosure, which do not depart from the instant disclosure. Hence, the following specification is intended to illustrate some particular embodiments of the disclosure, and not to exhaustively specify all permutations, combinations, and variations thereof.

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

### DEFINITIONS

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

The terminology used herein is for the purpose of describing particular embodiments only and is not intended to be limiting. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, elements, and/or components, but do not preclude the presence or addition of one or more other features, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

The term “about,” as used herein when referring to a measurable value such as an amount of a compound or agent of this disclosure, dose, time, temperature, and the like, is meant to encompass variations of ±10%, ±5%, ±1%, ±0.5%, or even ±0.10% of the specified amount.

As used herein, the transitional phrase “consisting essentially of’ is to be interpreted as encompassing the recited materials or steps and those that do not materially affect the basic and novel characteristic(s) of the disclosure. Thus, the term “consisting essentially of’ as used herein should not be interpreted as equivalent to “comprising.”

As used herein, the compositions described in the present disclosure are referred to interchangeably as (the singular or plural forms of) “nutritional compositions substantially similar to human milk,” “milk products,” “milk compositions,” “cultured milk products,” or equivalent as made clear by the context and mean the product secreted by the apical surface of a live cell construct (or, cell culture) comprising human mammary epithelial cells (hMEC). In some embodiments, the live cell construct is cultured in a bioreactor.

As used herein, the term “nanofiber” refers to fibers having a diameter or thickness in the nanometer range. For example, nanofibers may have a diameters or thicknesses ranging from about 0.1 nm to about 100000 nm, including from about 1 nm to about 1000 nm.

The term “polarized” as used herein in reference to cells and/or monolayers of cells refers to a spatial status of the cell wherein there are two distinct surfaces of the cell, e.g., an apical surface and a basal surface, which may be different. In some embodiments, the distinct surfaces of a polarized cell comprise different surface and/or transmembrane receptors and/or other structures. Individual polarized cells in a continuous monolayer may have similarly oriented apical surfaces and basal surfaces. Individual polarized cells in a continuous monolayer may have communicative structures between individual cells (e.g., tight junctions) to allow cross communication between individual cells and to create separation (e.g., compartmentalization) of the apical compartment and basal compartment.

As used herein, “apical surface” means the surface of a cell that faces an external environment or toward a cavity or chamber, for example the cavity of an internal organ. With respect to mammary epithelial cells, the apical surface is the surface from which the cultured milk product is secreted.

As used herein, “basal surface” means the surface of a cell that is in contact with a surface, e.g., the matrix of a bioreactor.

As used herein, “bioreactor” means a device or system that supports a biologically active environment that enables the production of a cultured milk product described herein from mammary cells described herein.

The term “lactogenic” as used herein refers to the ability to stimulate production and/or secretion of milk. A gene or protein (e.g., prolactin) may be lactogenic, as may any other natural and/or synthetic product. In some embodiments, a lactogenic culture medium comprises prolactin, thereby stimulating production of milk by cells in contact with the culture medium.

As used herein, the term “food grade” refers to materials considered non-toxic and safe for consumption (e.g., human and/or other animal consumption), e.g., as regulated by standards set by the U.S. Food and Drug Administration.

### Osteopontin Compositions

Osteopontin (OPN) is a protein found in human milk that has been implicated in immune regulation in breastfed infants, and a variety of other processes, including bone remineralization, tissue remodeling, gut health, and neurological development. OPN is produced by several types of cells throughout the body and there are multiple variants of the protein. Mammary epithelial cells produce a specific form of OPN with structural features that are essential for its activity. Mammary epithelial cells support a variety of structural modifications of OPN that may also contribute to the protein's function in the body. Improved recombinant osteopontin and methods for osteopontin production are needed.

Osteopontin (UniProt: P10451.1) is a protein found in human milk that is about 35 kDa in size and comprises an amino acid sequence of SEQ ID NO: 1 or SEQ ID NO: 2.

SEQ ID NO 1:
MRIAVICFCL LGITCAIPVK QADSGSSEEK QLYNKYPDAV
ATWLNPDPSQ KQNLLAPQNA VSSEETNDFK QETLPSKSNE
SHDHMDDMDD EDDDDHVDSQ DSIDSNDSDD VDDTDDSHQS
DESHHSDESD ELVTDFPTDL PATEVFTPVV PTVDTYDGRG
DSVVYGLRSK SKKFRRPDIQ YPDATDEDIT SHMESEELNG
AYKAIPVAQD LNAPSDWDSR GKDSYETSQL DDQSAETHSH
KQSRLYKRKA NDESNEHSDV IDSQELSKVS REFHSHEFHS
HEDMLVVDPK SKEEDKHLKF RISHELDSAS SEVN

SEQ ID NO 2:
MRIAVICGCL LGITCAIPVK QADSGSSEEK QLYNKYPDAV
ATWLNPDPSQ KQNLLAPQNA VSSEETNDFK QETLPSKSNE
SHDHMDDMDD EDDDDHVDSQ DSIDSNDSDD VDDTDDSHQS
DESHHSDESD ELVTDFPTDL PATEVFTPVV PTVDTYDGRG
DSVVYGLRSK SKKFRRPDIQ YPDATDEDIT SHMESEELNG
AYKAIPVAQD LNAPSDWDSR GKDSYETSQL DDQSAETHSH
KQSRLYKRKA NDESNEHSDV IDSQELSKVS REFHSHEFHS
HEDMLVVDPK SKEEDKHLKF RISHELDSAS SEVN

Described herein, in certain embodiments is a recombinant osteopontin comprising one or more post translational modification sites at positions 33, 36, 42, 66, 73, 237, 258, 267, 270, 291, 308, or 311 of SEQ ID NO: 1 or SEQ ID NO: 2. In some embodiments, the recombinant osteopontin comprises post translational modification sites at positions 33, 36, 42, 66, 73, 237, 258, 267, 270, 291, 308, and 311. In some embodiments, the recombinant osteopontin is phosphorylated at one or more post translational modification sites at positions 33, 36, 42, 66, 73, 237, 258, 267, 270, 291, 308, or 311 of SEQ ID NO: 1 or SEQ ID NO: 2.

In some embodiments, the post-translational modification is a chemical alteration. In some embodiments, the post-translational modification occurs at a functional group that can serve as a nucleophile in a reaction. In some embodiments, the functional group is selected from the group consisting of: hydroxyl group serine, threonine, and tyrosine; the amine forms of lysine, arginine, and histidine; the thiolate anion of cysteine; the carboxylates of aspartate and glutamate. In some embodiments, the post-translational modification occurs at a functional group with weak nucleophilic reactivity. In some embodiments, the functional group is selected from the group consisting of amide group asparagine; oxidized methionine; and methylene groups in amino acid side chains. In some embodiments, the post-translational modification comprises addition of a hydrophobic functional group. In some embodiments, the post-translational modification comprises addition of a hydrophobic functional group selected from the group consisting of myristoylation, palmitoylation, glypiation, geranylgeranylation, and farnesylation.

In some embodiments, the post-translational modification is selected from the group consisting of diphthamide formation, ethanolamine phosphoglycerol attachment, hypusine formation, and beta-Lysine addition. In some embodiments, the post-translational modification is selected from the group consisting of: acylation, acetylation, alkylation, amidation at the C-terminus, amide bond formation, amino acid addition, ADP-ribosylation, arginylation, butyrylation, formylation, gamma-carboxylation, glycosylation, hydroxylation, iodination, malonylation, methylation, nucleotide addition, phosphate ester formation, phosphoramidate formation, phosphorylation, polyglutamylation, polyglycylation, adenylylation, uridylylation, propionylation, pyroglutamate formation, S-glutathionylation, S-nitrosylation, S-sulfenylation, S-sulfonylation, succinylation, sulfation, sulfonation, biotinylation, carbamylation, carbonylation, glycation, spontaneous isopeptide bond formation, oxidation, pegylation. In some embodiments, the post-translational modification comprises chemical modification of amino acids and is selected from the group consisting of: citrullination, deamidation, and eliminylation.

In some embodiments, the post-translational modification comprises addition of an enzymatic cofactor. In some embodiments, the cofactor is selected from the group consisting of: lipoic acid, flavin, heme C, phosphopantetheine, and retinal.

In some embodiments, the post-translational modification comprises proteolytic cleavage (hydrolysis) of a peptide or isopeptide bond. In some embodiments, proteolytic cleavage removes the initiator methionine residue. In some embodiments, proteolytic cleavage produces a mature form protein, peptide, or polypeptide from a propeptide.

In some embodiments, the post-translational modification comprises structural changes and is selected from the group consisting of disulfide bridge formation, proteolytic cleavage, isoaspartate formation, racemization, and protein splicing. In some embodiments, the post-translational modification comprises conjugation with other proteins or peptides. In some embodiments, the post-translational modification is selected from the group consisting of ubiquitination, SUMOylation, neddylation, ISGylation, pupylation. In some embodiments, the post-translational modification is selected from the group consisting of: ubiquitination, acylation, isoprenylation, prenylation, alkylation, glycosylation, phosphorylation, and a disulfide bridge. In some embodiments, the post-translational modification is glycosylation. In some embodiments, the post-translational modification is a disulfide bridge.

In some embodiments, the recombinant osteopontin comprises a sequence of SEQ ID NO: 1 or SEQ ID NO: 2, wherein no more than 50 post translational modification sites in SEQ ID NO: 1 or SEQ ID NO: 2 are modified (e.g., phosphorylated or glycosylated). In some embodiments, the recombinant osteopontin comprises a sequence of SEQ ID NO: 1 or SEQ ID NO: 2, wherein no more than 45 post translational modification sites in SEQ ID NO: 1 or SEQ ID NO: 2 are modified. In some embodiments, the recombinant osteopontin comprises a sequence of SEQ ID NO: 1 or SEQ ID NO: 2, wherein no more than 40 post translational modification sites in SEQ ID NO: 1 or SEQ ID NO: 2 are modified. In some embodiments, the recombinant osteopontin comprises a sequence of SEQ ID NO: 1 or SEQ ID NO: 2, wherein no more than 35 post translational modification sites in SEQ ID NO: 1 or SEQ ID NO: 2 are modified. In some embodiments, the recombinant osteopontin comprises a sequence of SEQ ID NO: 1 or SEQ ID NO: 2, wherein no more than 30 post translational modification sites in SEQ ID NO: 1 or SEQ ID NO: 2 are modified. In some embodiments, the recombinant osteopontin comprises a sequence of SEQ ID NO: 1 or SEQ ID NO: 2, wherein no more than 25 post translational modification sites in SEQ ID NO: 1 or SEQ ID NO: 2 are modified. In some embodiments, the recombinant osteopontin comprises a sequence of SEQ ID NO: 1 or SEQ ID NO: 2, wherein no more than 20 post translational modification sites in SEQ ID NO: 1 or SEQ ID NO: 2 are modified. In some embodiments, the recombinant osteopontin comprises a sequence of SEQ ID NO: 1 or SEQ ID NO: 2, wherein no more than 15 post translational modification sites in SEQ ID NO: 1 or SEQ ID NO: 2 are modified.

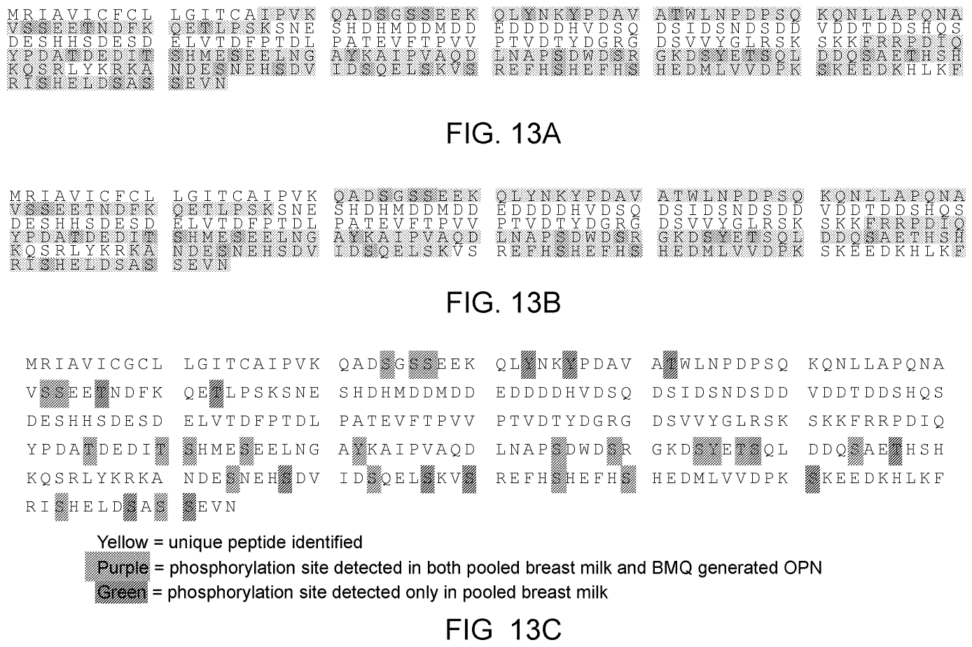

In some embodiments, the recombinant osteopontin comprises one or more peptide sequences. See FIG. 13C. In some embodiments, the one or more peptide sequences comprises one or more phosphorylation sites. In some embodiments, the one or more peptide sequences comprises at least 1, 2, 3, 4, 5, or more than 5 phosphorylation sites. In some embodiments, the recombinant osteopontin comprises one or more peptide sequences that are isomers of each other.

In some embodiments, the recombinant osteopontin is about 30 to about 40 kDa in size. In some embodiments, recombinant osteopontin is about 31 to about 39 kDa in size. In some embodiments, recombinant osteopontin is about 32 to about 38 kDa in size. In some embodiments, recombinant osteopontin is about 33 to about 37 kDa in size. In some embodiments, recombinant osteopontin is about 34 to about 39 kDa in size. In some embodiments, recombinant osteopontin is about 35 kDa in size.

In some embodiments, the recombinant osteopontin is about 30 to about 75 kDa in size. In some embodiments, recombinant osteopontin is about 35 to about 75 kDa in size. In some embodiments, recombinant osteopontin is about 40 to about 75 kDa in size. In some embodiments, recombinant osteopontin is about 45 to about 75 kDa in size. In some embodiments, recombinant osteopontin is about 50 to about 75 kDa in size. In some embodiments, recombinant osteopontin is about 55 to about 75 kDa in size. In some embodiments, recombinant osteopontin is about 60 to about 75 kDa in size. In some embodiments, recombinant osteopontin is about 65 to about 75 kDa in size. In some embodiments, recombinant osteopontin is about 70 to about 75 kDa in size. In some embodiments, recombinant osteopontin is about 32 kDa in size. In some embodiments, recombinant osteopontin is about 34 kDa in size. In some embodiments, recombinant osteopontin is about 36 kDa in size. In some embodiments, recombinant osteopontin is about 38 kDa in size. In some embodiments, recombinant osteopontin is about 40 kDa in size. In some embodiments, recombinant osteopontin is about 42 kDa in size. In some embodiments, recombinant osteopontin is about 44 kDa in size. In some embodiments, recombinant osteopontin is about 46 kDa in size. In some embodiments, recombinant osteopontin is about 48 kDa in size. In some embodiments, recombinant osteopontin is about 50 kDa in size. In some embodiments, recombinant osteopontin is about 52 kDa in size. In some embodiments, recombinant osteopontin is about 54 kDa in size. In some embodiments, recombinant osteopontin is about 56 kDa in size. In some embodiments, recombinant osteopontin is about 58 kDa in size. In some embodiments, recombinant osteopontin is about 60 kDa in size. In some embodiments, recombinant osteopontin is about 62 kDa in size. In some embodiments, recombinant osteopontin is about 64 kDa in size. In some embodiments, recombinant osteopontin is about 66 kDa in size. In some embodiments, recombinant osteopontin is about 68 kDa in size. In some embodiments, recombinant osteopontin is about 70 kDa in size. In some embodiments, recombinant osteopontin is about 72 kDa in size. In some embodiments, recombinant osteopontin is about 74 kDa in size.

Further described herein, in certain embodiments, are methods of use of recombinant osteopontin described herein. In some embodiments, the recombinant osteopontin is used for immune regulation, anti-bacterial activity, bone remineralization, tissue remodeling, gut health, neurological development, organ development, wound healing, or stroke recovery. In some embodiments, the recombinant osteopontin is used for immune regulation. In some embodiments, the recombinant osteopontin is used for anti-bacterial activity. In some embodiments, the recombinant osteopontin is used for bone remineralization. In some embodiments, the recombinant osteopontin is used for tissue remodeling. In some embodiments, the recombinant osteopontin is used for gut health. In some embodiments, the recombinant osteopontin is used for neurological development. In some embodiments, the recombinant osteopontin is used for organ development. In some embodiments, the recombinant osteopontin is used for wound healing. In some embodiments, the recombinant osteopontin is used for stroke recovery.

### Methods of Producing Osteopontin

Described herein, in certain embodiments, are methods of producing recombinant osteopontin. In some embodiments, the method comprises a method of producing osteopontin from mammary cells. In some embodiments, the method of producing recombinant osteopontin from mammary cells comprises: (a) culturing a live cell construct in a bioreactor under conditions which produce a cultured milk product, said live cell construct comprising: (i) a three-dimensional scaffold having an exterior surface, an interior surface defining an interior cavity/basal chamber, and a plurality of pores extending from the interior surface to the exterior surface; (ii) a matrix material disposed on the exterior surface of the three-dimensional scaffold; (ii) a culture media disposed within the interior cavity/basal chamber and in fluidic contact with the internal surface; and (iv) an at least 70% confluent monolayer of polarized mammary cells disposed on the matrix material, wherein the mammary cells are modified to overexpress the recombinant osteopontin; and (b) isolating the recombinant osteopontin from the cultured milk product.

In some embodiments, osteopontin is expressed as a recombinant form of osteopontin. In some embodiments, osteopontin is post-translationally modified. In some embodiments, the post-translational modification comprises glycosylation or phosphorylation. In some embodiments, the post-translational modification comprises glycosylation. In some embodiments, the post-translational modification comprises phosphorylation. In some embodiments, enzymes within the mammary cell carry out post-translational modification of osteopontin.

In some embodiments of the method, the mammary cells are modified to overexpress the recombinant osteopontin by expressing a vector comprising a polynucleotide having least 80% identity to a sequence as set forth in SEQ ID NO: 3. In some embodiments, the mammary cells are modified to overexpress the recombinant osteopontin by expressing a vector comprising a polynucleotide having least 90% identity to a sequence as set forth in SEQ ID NO: 3. In some embodiments, the mammary cells are modified to overexpress the recombinant osteopontin by expressing a vector comprising a polynucleotide having least 95% identity to a sequence as set forth in SEQ ID NO: 3. In some embodiments, the mammary cells are modified to overexpress the recombinant osteopontin by expressing a vector comprising a polynucleotide having least 96% identity to a sequence as set forth in SEQ ID NO: 3. In some embodiments, the mammary cells are modified to overexpress the recombinant osteopontin by expressing a vector comprising a polynucleotide having least 97% identity to a sequence as set forth in SEQ ID NO: 3. In some embodiments, the mammary cells are modified to overexpress the recombinant osteopontin by expressing a vector comprising a polynucleotide having least 98% identity to a sequence as set forth in SEQ ID NO: 3. In some embodiments, the mammary cells are modified to overexpress the recombinant osteopontin by expressing a vector comprising a polynucleotide having least 99% identity to a sequence as set forth in SEQ ID NO: 3. In some embodiments, the mammary cells are modified to overexpress the recombinant osteopontin by expressing a vector comprising a polynucleotide having least 99.5% identity to a sequence as set forth in SEQ ID NO: 3. In some embodiments, the mammary cells are modified to overexpress the recombinant osteopontin by expressing a vector comprising a polynucleotide having a sequence as set forth in SEQ ID NO: 3.

| SEQ | | |
|---|---|---|
| ID | | |
| NO | Description | Sequence |
| 3 | Vector | TTAACCCTAGAAAGATAGTCTGCGTAAAATTGACGCATGCATTC |
| | | TTGAAATATTGCTCTCTCTTTCTAAATAGCGCGAATCCGTCGCT |
| | | GTGCATTTAGGACATCTCAGTCGCCGCTTGGAGCTCCCGTGAGG |
| | | CGTGCTTGTCAATGCGGTAAGTGTCACTGATTTTGAACTATAAC |
| | | GACCGCGTGAGTCAAAATGACGCATGATTATCTTTTACGTGACT |
| | | TTTAAGATTTAACTCATACGATAATTATATTGTTATTTCATGTT |
| | | CTACTTACGTGATAACTTATTATATATATATTTTCTTGTTATAG |
| | | ATATCATCAACTTTGTATAGAAAAGTTGGGCTCCGGTGCCCGTC |
| | | AGTGGGCAGAGCGCACATCGCCCACAGTCCCCGAGAAGTTGGGG |
| | | GGAGGGGTCGGCAATTGAACCGGTGCCTAGAGAAGGTGGCGCGG |
| | | GGTAAACTGGGAAAGTGATGTCGTGTACTGGCTCCGCCTTTTTC |
| | | CCGAGGGTGGGGGAGAACCGTATATAAGTGCAGTAGTCGCCGTG |
| | | AACGTTCTTTTTCGCAACGGGTTTGCCGCCAGAACACAGGTAAG |
| | | TGCCGTGTGTGGTTCCCGCGGGCCTGGCCTCTTTACGGGTTATG |
| | | GCCCTTGCGTGCCTTGAATTACTTCCACCTGGCTGCAGTACGTG |
| | | ATTCTTGATCCCGAGCTTCGGGTTGGAAGTGGGTGGGAGAGTTC |
| | | GAGGCCTTGCGCTTAAGGAGCCCCTTCGCCTCGTGCTTGAGTTG |
| | | AGGCCTGGCCTGGGCGCTGGGGCCGCCGCGTGCGAATCTGGTGG |
| | | CACCTTCGCGCCTGTCTCGCTGCTTTCGATAAGTCTCTAGCCAT |
| | | TTAAAATTTTTGATGACCTGCTGCGACGCTTTTTTTCTGGCAAG |
| | | ATAGTCTTGTAAATGCGGGCCAAGATCTGCACACTGGTATTTCG |
| | | GTTTTTGGGGCCGCGGGCGGCGACGGGGCCCGTGCGTCCCAGCG |
| | | CACATGTTCGGCGAGGCGGGGCCTGCGAGCGCGGCCACCGAGAA |
| | | TCGGACGGGGGTAGTCTCAAGCTGGCCGGCCTGCTCTGGTGCCT |
| | | GGTCTCGCGCCGCCGTGTATCGCCCCGCCCTGGGCGGCAAGGCT |
| | | GGCCCGGTCGGCACCAGTTGCGTGAGCGGAAAGATGGCCGCTTC |
| | | CCGGCCCTGCTGCAGGGAGCTCAAAATGGAGGACGCGGCGCTCG |
| | | GGAGAGCGGGCGGGTGAGTCACCCACACAAAGGAAAAGGGCCTT |
| | | TCCGTCCTCAGCCGTCGCTTCATGTGACTCCACGGAGTACCGGG |
| | | CGCCGTCCAGGCACCTCGATTAGTTCTCGAGCTTTTGGAGTACG |
| | | TCGTCTTTAGGTTGGGGGGAGGGGTTTTATGCGATGGAGTTTCC |
| | | CCACACTGAGTGGGTGGAGACTGAAGTTAGGCCAGCTTGGCACT |
| | | TGATGTAATTCTCCTTGGAATTTGCCCTTTTTGAGTTTGGATCT |
| | | TGGTTCATTCTCAAGCCTCAGACAGTGGTTCAAAGTTTTTTTCT |
| | | TCCATTTCAGGTGTCGTGACAAGTTTGTACAAAAAAGCAGGCTG |
| | | CCACCATGAGAATCGCCGTGATCTGTTTCTGTCTGCTGGGCATC |
| | | ACCTGTGCCATTCCCGTCAAGCAGGCCGACTCCGGCTCTAGCGA |
| | | GGAGAAGCAGCTGTATAATAAATACCCCGACGCCGTGGCTACCT |
| | | GGCTGAACCCCGACCCAAGCCAGAAACAGAACCTGCTGGCCCCC |
| | | CAGAACGCCGTGTCCAGTGAGGAGACCAATGATTTCAAGCAGGA |
| | | GACCCTGCCCTCCAAAAGCAACGAAAGCCACGACCACATGGATG |
| | | ACATGGATGACGAGGACGATGATGATCACGTGGACTCACAGGAC |
| | | AGCATCGACTCCAACGACAGCGACGACGTGGACGATACCGACGA |
| | | TTCACACCAGTCTGACGAGAGCCACCACTCCGACGAGAGCGACG |
| | | AACTGGTGACCGACTTTCCCACAGACCTGCCTGCTACCGAGGTG |
| | | TTTACCCCAGTGGTCCCCACTGTGGATACCTACGACGGGCGGGG |
| | | CGACAGCGTGGTCTACGGCCTGCGGTCCAAGTCTAAAAAATTTA |
| | | GGAGGCCCGATATTCAGTACCCCGACGCCACTGACGAAGATATT |
| | | ACTTCACATATGGAGAGCGAGGAGCTGAATGGCGCCTACAAGGC |
| | | CATCCCCGTGGCTCAGGACCTGAACGCTCCTAGCGATTGGGACA |
| | | GCAGAGGCAAGGATAGCTACGAGACCAGTCAGCTGGACGACCAG |
| | | TCTGCCGAGACCCATTCTCACAAGCAGAGCCGCCTGTACAAGCG |
| | | GAAGGCTAACGACGAGAGCAACGAGCACTCAGACGTGATTGACT |
| | | CCCAGGAGCTGAGCAAAGTGTCTCGGGAGTTTCACTCTCACGAA |
| | | TTCCACTCCCACGAGGACATGCTGGTGGTGGACCCCAAAAGCAA |
| | | GGAGGAGGATAAGCATCTGAAATTCCGGATCAGTCACGAGCTGG |
| | | ATTCCGCCTCCAGCGAGGTGAACGGAAGCGGAGCCACGAACTTC |
| | | TCTCTGTTAAAGCAAGCAGGAGATGTTGAAGAAAACCCCGGGCC |
| | | TATGGCCAAGCCTTTGTCTCAAGAAGAATCCACCCTCATTGAAA |
| | | GAGCAACGGCTACAATCAACAGCATCCCCATCTCTGAAGACTAC |
| | | AGCGTCGCCAGCGCAGCTCTCTCTAGCGACGGCCGCATCTTCAC |
| | | TGGTGTCAATGTATATCATTTTACTGGGGGACCTTGTGCAGAAC |
| | | TCGTGGTGCTGGGCACTGCTGCTGCTGCGGCAGCTGGCAACCTG |
| | | ACTTGTATCGTCGCGATCGGAAATGAGAACAGGGGCATCTTGAG |
| | | CCCCTGCGGACGGTGCCGACAGGTGCTTCTCGATCTGCATCCTG |
| | | GGATCAAAGCCATAGTGAAGGACAGTGATGGACAGCCGACGGCA |
| | | GTTGGGATTCGTGAATTGCTGCCCTCTGGTTATGTGTGGGAGGG |
| | | CTAAACCCAGCTTTCTTGTACAAAGTGGTGATCCTCAGGTGCAG |
| | | GCTGCCTATCAGAAGGTGGTGGCTGGTGTGGCCAATGCCCTGGC |
| | | TCACAAATACCACTGAGATCTTTTTCCCTCTGCCAAAAATTATG |
| | | GGGACATCATGAAGCCCCTTGAGCATCTGACTTCTGGCTAATAA |
| | | AGGAAATTTATTTTCATTGCAATAGTGTGTTGGAATTTTTTGTG |
| | | TCTCTCACTCGGAAGGACATATGGGAGGGCAAATCATTTAAAAC |
| | | ATCAGAATGAGTATTTGGTTTAGAGTTTGGCAACATATGCCCAT |
| | | ATGCTGGCTGCCATGAACAAAGGTTGGCTATAAAGAGGTCATCA |
| | | GTATATGAAACAGCCCCCTGCTGTCCATTCCTTATTCCATAGAA |
| | | AAGCCTTGACTTGAGGTTAGATTTTTTTTATATTTTGTTTTGTG |
| | | TTATTTTTTTCTTTAACATCCCTAAAATTTTCCTTACATGTTTT |
| | | ACTAGCCAGATTTTTCCTCCTCTCCTGACTACTCCCAGTCATAG |
| | | CTGTCCCTCTTCTCTTATGGAGATCCCTCGACCTGCAGCCCAAG |
| | | CTTGGATCCCTCGAGTTAATTAACGAGAGCATAATATTGATATG |
| | | TGCCAAAGTTGTTTCTGACTGACTAATAAGTATAATTTGTTTCT |
| | | ATTATGTATAGGTTAAGCTAATTACTTATTTTATAATACAACAT |
| | | GACTGTTTTTAAAGTACAAAATAAGTTTATTTTTGTAAAAGAGA |
| | | GAATGTTTAAAAGTTTTGTTACTTTATAGAAGAAATTTTGAGTT |
| | | TTTGTTTTTTTTTAATAAATAAATAAACATAAATAAATTGTTTG |
| | | TTGAATTTATTATTAGTATGTAAGTGTAAATATAATAAAACTTA |
| | | ATATCTATTCAAATTAATAAATAAACCTCGATATACAGACCGAT |
| | | AAAACACATGCGTCAATTTTACGCATGATTATCTTTAACGTACG |
| | | TCACAATATGATTATCTTTCTAGGGTTAAATAATAGTTTCTAAT |
| | | TTTTTTATTATTCAGCCTGCTGTCGTGAATACCGAGCTCCAATT |
| | | CGCCCTATAGTGAGTCGTATTACAATTCACTGGCCGTCGTTTTA |
| | | CAACGTCGTGACTGGGAAAACCCTGGCGTTACCCAACTTAATCG |
| | | CCTTGCAGCACATCCCCCTTTCGCCAGCTGGCGTAATAGCGAAG |
| | | AGGCCCGCACCGATCGCCCTTCCCAACAGTTGCGCAGCCTGAAT |
| | | GGCGAATGGGACGCGCCCTGTAGCGGCGCATTAAGCGCGGCGGG |
| | | TGTGGTGGTTACGCGCAGCGTGACCGCTACACTTGCCAGCGCCC |
| | | TAGCGCCCGCTCCTTTCGCTTTCTTCCCTTCCTTTCTCGCCACG |
| | | TTCGCCGGCTTTCCCCGTCAAGCTCTAAATCGGGGGCTCCCTTT |
| | | AGGGTTCCGATTTAGTGCTTTACGGCACCTCGACCCCAAAAAAC |
| | | TTGATTAGGGTGATGGTTCACGTAGTGGGCCATCGCCCTGATAG |
| | | ACGGTTTTTCGCCCTTTGACGTTGGAGTCCACGTTCTTTAATAG |
| | | TGGACTCTTGTTCCAAACTGGAACAACACTCAACCCTATCTCGG |
| | | TCTATTCTTTTGATTTATAAGGGATTTTGCCGATTTCGGCCTAT |
| | | TGGTTAAAAAATGAGCTGATTTAACAAAAATTTAACGCGAATTT |
| | | TAACAAAATATTAACGCTTACAATTTAGGTGGCACTTTTCGGGG |
| | | AAATGTGCGCGGAACCCCTATTTGTTTATTTTTCTAAATACATT |
| | | CAAATATGTATCCGCTCATGAGACAATAACCCTGATAAATGCTT |
| | | CAATAATATTGAAAAAGGAAGAGTATGAGTATTCAACATTTCCG |
| | | TGTCGCCCTTATTCCCTTTTTTGCGGCATTTTGCCTTCCTGTTT |
| | | TTGCTCACCCAGAAACGCTGGTGAAAGTAAAAGATGCTGAAGAT |
| | | CAGTTGGGTGCACGAGTGGGTTACATCGAACTGGATCTCAACAG |
| | | CGGTAAGATCCTTGAGAGTTTTCGCCCCGAAGAACGTTTTCCAA |
| | | TGATGAGCACTTTTAAAGTTCTGCTATGTGGCGCGGTATTATCC |
| | | CGTATTGACGCCGGGCAAGAGCAACTCGGTCGCCGCATACACTA |
| | | TTCTCAGAATGACTTGGTTGAGTACTCACCAGTCACAGAAAAGC |
| | | ATCTTACGGATGGCATGACAGTAAGAGAATTATGCAGTGCTGCC |
| | | ATAACCATGAGTGATAACACTGCGGCCAACTTACTTCTGACAAC |
| | | GATCGGAGGACCGAAGGAGCTAACCGCTTTTTTGCACAACATGG |
| | | GGGATCATGTAACTCGCCTTGATCGTTGGGAACCGGAGCTGAAT |
| | | GAAGCCATACCAAACGACGAGCGTGACACCACGATGCCTGTAGC |
| | | AATGGCAACAACGTTGCGCAAACTATTAACTGGCGAACTACTTA |
| | | CTCTAGCTTCCCGGCAACAATTAATAGACTGGATGGAGGCGGAT |
| | | AAAGTTGCAGGACCACTTCTGCGCTCGGCCCTTCCGGCTGGCTG |
| | | GTTTATTGCTGATAAATCTGGAGCCGGTGAGCGTGGGTCTCGCG |
| | | GTATCATTGCAGCACTGGGGCCAGATGGTAAGCCCTCCCGTATC |
| | | GTAGTTATCTACACGACGGGGAGTCAGGCAACTATGGATGAACG |
| | | AAATAGACAGATCGCTGAGATAGGTGCCTCACTGATTAAGCATT |
| | | GGTAACTGTCAGACCAAGTTTACTCATATATACTTTAGATTGAT |
| | | TTAAAACTTCATTTTTAATTTAAAAGGATCTAGGTGAAGATCCT |
| | | TTTTGATAATCTCATGACCAAAATCCCTTAACGTGAGTTTTCGT |
| | | TCCACTGAGCGTCAGACCCCGTAGAAAAGATCAAAGGATCTTCT |
| | | TGAGATCCTTTTTTTCTGCGCGTAATCTGCTGCTTGCAAACAAA |
| | | AAAACCACCGCTACCAGCGGTGGTTTGTTTGCCGGATCAAGAGC |
| | | TACCAACTCTTTTTCCGAAGGTAACTGGCTTCAGCAGAGCGCAG |
| | | ATACCAAATACTGTTCTTCTAGTGTAGCCGTAGTTAGGCCACCA |
| | | CTTCAAGAACTCTGTAGCACCGCCTACATACCTCGCTCTGCTAA |
| | | TCCTGTTACCAGTGGCTGCTGCCAGTGGCGATAAGTCGTGTCTT |
| | | ACCGGGTTGGACTCAAGACGATAGTTACCGGATAAGGCGCAGCG |
| | | GTCGGGCTGAACGGGGGGTTCGTGCACACAGCCCAGCTTGGAGC |
| | | GAACGACCTACACCGAACTGAGATACCTACAGCGTGAGCTATGA |
| | | GAAAGCGCCACGCTTCCCGAAGAGAGAAAGGCGGACAGGTATCC |
| | | GGTAAGCGGCAGGGTCGGAACAGGAGAGCGCACGAGGGAGCTTC |
| | | CAGGGGGAAACGCCTGGTATCTTTATAGTCCTGTCGGGTTTCGC |
| | | CACCTCTGACTTGAGCGTCGATTTTTGTGATGCTCGTCAGGGGG |
| | | GCGGAGCCTATGGAAAAACGCCAGCAACGCGGCCTTTTTACGGT |
| | | TCCTGGCCTTTTGCTGGCCTTTTGCTCACATGTTCTTTCCTGCG |
| | | TTATCCCCTGATTCTGTGGATAACCGTATTACCGCCTTTGAGTG |
| | | AGCTGATACCGCTCGCCGCAGCCGAACGACCGAGCGCAGCGAGT |
| | | CAGTGAGCGAGGAAGCGGAAGAGCGCCCAATACGCAAACCGCCT |
| | | CTCCCCGCGCGTTGGCCGATTCATTAATGCAGCTGGCACGACAG |
| | | GTTTCCCGACTGGAAAGCGGGCAGTGAGCGCAACGCAATTAATG |
| | | TGAGTTAGCTCACTCATTAGGCACCCCAGGCTTTACACTTTATG |
| | | CTTCCGGCTCGTATGTTGTGTGGAATTGTGAGCGGATAACAATT |
| | | TCACACAGGAAACAGCTATGACCATGATTACGCCAAGCTCGAAA |
| | | TTAACCCTCACTAAAGGGAACAAAAGCTGGTACCTCGCGCGACT |
| | | TGGTTTGCCATTCTTTAGCGCGCGTCGCGTCACACAGCTTGGCC |
| | | ACAATGTGGTTTTTGTCAAACGAAGATTCTATGACGTGTTTAAA |
| | | GTTTAGGTCGAGTAAAGCGCAAATCTTTT |

In some embodiments, the vector is a plasmid (e.g., circular DNA molecules that can autonomously replicate inside a cell), cosmid (e.g., pWE or sCos vectors), artificial chromosome, human artificial chromosome (HAC), yeast artificial chromosomes (YAC), bacterial artificial chromosome (BAC), P1-derived artificial chromosomes (PAC), phagemid, phage derivative, bacmid, or virus. In some embodiments, the vector is selected from the list consisting of pSF-CMV-NEO-NH-2-PPT-3XFLAG, pSF-CMV-NEO-COOH-3XFLAG, pSF-CMV-PURO-NH2-GST-TEV, pSF-OXB20-COOH-TEV-FLAG(R)-6His, pCEP4 pDEST27, pSF-CMV-Ub-KrYFP, pSF-CMV-FMDV-daGFP, pEF1a-mCherry-N1 vector, pEF1a-tdTomato vector, pSF-CMV-FMDV-Hygro, pSF-CMV-PGK-Puro, pMCP-tag(m), pSF-CMV-PURO-NH2-CMYC, pSF-OXB20-BetaGal, pSF-OXB20-Fluc, pSF-OXB20, pSF-Tac, pRI 101-AN DNA, pCambia2301, pTYB21, pKLAC2, pAc5.1/V5-His A, and pDEST8.

In some embodiments, the vector comprises a promoter. In some embodiments, the promoter is selected from the group consisting of a mini promoter, an inducible promoter, a constitutive promoter, and derivatives thereof. In some embodiments, the promoter is selected from the group consisting of CMV, CBA, EF1a, CAG, PGK, TRE, U6, UAS, T7, Sp6, lac, araBad, trp, Ptac, p5, p19, p40, Synapsin, CaMKII, GRK1, and derivatives thereof.

In some embodiments, the vector comprises inverted terminal repeats. In some embodiments, the vector comprises long terminal repeats. In some embodiments, the vector comprises a translation initiation sequence. In some embodiments the translation initiation sequence is a Kozak sequence. In some embodiments, the vector comprises an open reading frame.

In some embodiments, the vector comprises a linker. In some embodiments, the linker is a fusion linker, a 2A peptide linker, an internal ribosome entry site linker. In some embodiments, the linker is selected from the group consisting of 3xGGGGS (SEQ ID NO: 4), 3xGS (SEQ ID NO: 5), T2A, P2A, F2A, E2A, T2A(dme), P2a(dme), E2A(dme), IRES, Ubx-IRES, and derivatives thereof.

In some embodiments, the vector comprises a selection marker. In some embodiments the selection marker is selected from the group of: ampicillin, puromycin, hygromycin, neomycin, or blasticidin.

In some embodiments, the vector is a viral vector. In some embodiments, the virus is an alphavirus, a parvovirus, an adenovirus, an AAV, a baculovirus, a Dengue virus, a lentivirus, a herpesvirus, a poxvirus, an anellovirus, a bocavirus, a vaccinia virus, or a retrovirus. In some embodiments, the virus is an alphavirus. In some embodiments, the virus is a parvovirus. In some embodiments, the virus is an adenovirus. In some embodiments, the virus is an AAV. In some embodiments, the virus is a baculovirus. In some embodiments, the virus is a Dengue virus. In some embodiments, the virus is a lentivirus. In some embodiments, the virus is a herpesvirus. In some embodiments, the virus is a poxvirus. In some embodiments, the virus is an anellovirus. In some embodiments, the virus is a bocavirus. In some embodiments, the virus is a vaccinia virus. In some embodiments, the virus is or a retrovirus.

In some embodiments, the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, AAV-HSC16, or a derivative thereof. In some embodiments, the herpesvirus is HSV type 1, HSV-2, VZV, EBV, CMV, HHV-6, HHV-7, or HHV-8.

In some embodiments, the polynucleotide is provided in a non-viral delivery system. In some embodiments, the nucleic acid is comprised in a liposome. In some embodiments, the polynucleotide is associated with a lipid. The polynucleotide associated with a lipid, in some embodiments, is encapsulated in the aqueous interior of a liposome, interspersed within the lipid bilayer of a liposome, attached to a liposome via a linking molecule that is associated with both the liposome and the nucleic acid, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. In some embodiments, the polynucleotide is provided in a lipid nanoparticle (LNP).

Vectors can be introduced into a cell by a variety of methods, including transformation, transfection, direct uptake, projectile bombardment, and by encapsulation of the vector in a liposome or nanoparticle. Examples of suitable methods of transfecting or transforming cells are calcium phosphate precipitation, electroporation, microinjection, infection, lipofection, and direct uptake. Such methods are described in more detail, for example, in Green et al., Molecular Cloning: A Laboratory Manual, Fourth Edition (Cold Spring Harbor University Press, New York (2014)); and Ausubel et al., Current Protocols in Molecular Biology (John Wiley & Sons, New York (2015)).

In some embodiments, the mammary cells are selected from the group consisting of primary mammary epithelial cells, mammary myoepithelial cells, mammary progenitor cells, immortalized mammary epithelial cells, immortalized mammary myoepithelial cells, and immortalized mammary progenitor cells. In some embodiments, the mammary cells are primary mammary epithelial cells. In some embodiments, the mammary cells are mammary myoepithelial cells. In some embodiments, the mammary cells are mammary progenitor cells. In some embodiments, the mammary cells are immortalized mammary epithelial cells. In some embodiments, the mammary cells are immortalized mammary myoepithelial cells. In some embodiments, the mammary cells are immortalized mammary progenitor cells.

In some embodiments the mammary cell is a non-human cell. In some embodiments, the non-human cell are selected from a cow cell, a bison cell, a buffalo cell, a yak cell, a sheep cell, a goat cell, a pig cell, a reindeer cell, a horse cell, a dog cell, or a cat cell.

In some embodiments, the mammary cell is human.

In some embodiments, the polarized mammary cell comprises an apical and a basal surface. In some embodiments, the basal surface of the mammary cells is in fluidic contact with the culture media.

In some embodiments, the bioreactor is an enclosed bioreactor. In some embodiments, the bioreactor is selected from the group consisting of a stirred-tank bioreactor, a spinner flask bioreactor, a rotating wall bioreactor, a rocker bioreactor, an air lift bioreactor, a fixed-bed bioreactor, and a hollow fiber bioreactor.

In some embodiments, the bioreactor comprises an apical compartment that is substantially isolated from the internal cavity/basal chamber of the live cell construct. In some embodiments, the apical compartment is in fluidic contact with the apical surface of the mammary cells. In some embodiments, the cultured milk product is secreted from the apical surface of the mammary cells into the apical compartment. In some embodiments, the culture media substantially does not contact the cultured milk product.

In some embodiments, the total cell density of mammary cells within the bioreactor is at least 10⁵-10¹⁶. In some embodiments, the total cell density of mammary cells within the bioreactor is at least 10⁵. In some embodiments, the total cell density of mammary cells within the bioreactor is at least 10⁶. In some embodiments, the total cell density of mammary cells within the bioreactor is at least 10⁷. In some embodiments, the total cell density of mammary cells within the bioreactor is at least 10⁸. In some embodiments, the total cell density of mammary cells within the bioreactor is at least 10⁹. In some embodiments, the total cell density of mammary cells within the bioreactor is at least 10¹⁰. In some embodiments, the total cell density of mammary cells within the bioreactor is at least 10¹¹. In some embodiments, the total cell density of mammary cells within the bioreactor is at least 10¹². In some embodiments, the total cell density of mammary cells within the bioreactor is at least 10¹³. In some embodiments, the total cell density of mammary cells within the bioreactor is at least 10¹⁴. In some embodiments, the total cell density of mammary cells within the bioreactor is at least 10¹⁵.

In some embodiments, the total surface area of mammary cells within the bioreactor is at least 0.5 m², 1 m², 1.5 m², 2 m², 2.5 m², or 3 m². In some embodiments, the total surface area of mammary cells within the bioreactor is at least 0.5-3 m². In some embodiments, the total surface area of mammary cells within the bioreactor is at least 1-2.5 m². In some embodiments, the total surface area of mammary cells within the bioreactor is at least 1.5-2 m². In some embodiments, the total surface area of mammary cells within the bioreactor is at least 1.5 m².

In some embodiments, the culture medium comprises a carbon source, a chemical buffering system, one or more essential amino acids, one or more non-essential amino acids, one or more vitamins and/or cofactors, one or more organic compounds, one or more growth factors, one or more trace minerals, and one or more inorganic salts. In some embodiments, the culture medium comprises a carbon source, a chemical buffering system, one or more essential amino acids, one or more vitamins and/or cofactors, and one or more inorganic salts.

In some embodiments, the matrix material comprises one or more extracellular matrix proteins. In some embodiments, the scaffold comprises a natural polymer, a biocompatible synthetic polymer, a synthetic peptide, a composite derived from any of the preceding, or any combination thereof. In some embodiments, natural polymer is collagen, chitosan, cellulose, agarose, alginate, gelatin, elastin, heparan sulfate, chondroitin sulfate, keratan sulfate, and/or hyaluronic acid. In some embodiments, the biocompatible synthetic polymer is polysulfone, polyvinylidene fluoride, polyethylene co-vinyl acetate, polyvinyl alcohol, sodium polyacrylate, an acrylate polymer, and/or polyethylene glycol.

In some embodiments, the culturing is carried out at a temperature of about 27 °C to about 39 °C (e.g., a temperature of about 27 °C, 27.5 °C, 28 °C, 28.5 °C, 29 °C, 29.5 °C, 30 °C, 30.5 °C, 31 °C, 31.5 °C, 32 °C, 32.5 °C, 33 °C, 33.5 °C, 34 °C, 34.5 °C, 35 °C, 35.5 °C, 36 °C, 36.5 °C, 37 °C, 37.5 °C, 38 °C, 38.5 °C or about 39 °C, or any value or range therein, e.g., about 35 °C to about 38 °C, about 36 °C to about 39 °C, about 36.5 °C to about 39 °C, about 36.5 °C to about 37.5 °C, or about 36.5 °C to about 38 °C). In some embodiments, the culturing is carried out at a temperature of about 30 °C to about 37 °C (e.g., a temperature of about 30 °C, 30.5 °C, 31 °C, 31.5 °C, 32 °C, 32.5 °C, 33 °C, 33.5 °C, 34 °C, 34.5 °C, 35 °C, 35.5 °C, 36 °C, 36.5 °C, or about 37 °C, or any value or range therein, e.g., about 32 °C to about 35 °C, about 33 °C to about 36 °C, about 33.5 °C to about 36 °C, about 32.5 °C to about 35.5 °C, or about 34.5 °C to about 37 °C).

In some embodiments, the culturing is carried out at an atmospheric concentration of CO2 of about 3%, 3.25%, 3.5%, 3.75%, 4%, 4.25%, 4.5%, 4.75%, 5%, 5.25%, 5.5%, 5.75%, 6%, 6.25%, or 6.5% or any value or range therein, e.g., about 3% to about 4.5%, about 3.5% to about 5%, about 4% to about 5.5%, about 4.5% to about 6%, about 5% to about 6.5%, about 4% to about 5%, about 4.5% to about 5.5%, about 5% to about 6%, or about 5.5% to about 6.5%). In some embodiments, the culturing is carried out at an atmospheric concentration of CO2 of about 4% to about 6%. In some embodiments, the culturing is carried out at an atmospheric concentration of CO2 of about 5%.

### Bioreactors

Described herein, in certain embodiments, are recombinant osteopontin and methods of producing recombinant osteopontin. In some embodiments, methods for producing recombinant osteopontin comprises using a bioreactor.

In some embodiments, the bioreactor is an enclosed bioreactor. In some embodiments, the apical chamber is substantially isolated from the interior cavity/basal compartment. In some embodiments, the total cell density of mammary cells within the bioreactor is at least 10⁵, 10⁶, 10⁷, 10⁸, 10⁹, 10¹⁰, 10¹¹, 10¹², 10¹³, 10¹⁴, 10¹⁵, or 10¹⁶. In some embodiments, the total cell density of mammary cells within the bioreactor is at least 10¹¹. In some embodiments, the total cell density of mammary cells within the bioreactor is at least 0.5 m², 1 m², 1.5 m², 2 m², 2.5 m², or 3 m². In some embodiments, the total cell density of mammary cells within the bioreactor is at least 1.5 m².

A hollow fiber bioreactor is an exemplary bioreactor for use with the methods disclosed here. The hollow fiber bioreactor is a high-density, continuous perfusion culture system that closely approximates the environment in which cells grow in vivo. It consists of thousands of semi-permeable three-dimensional scaffolds (e.g., hollow tubes made up of a plurality of fibers, such as electrospun fibers), as described herein, in a parallel array within a cartridge shell fitted with inlet and outlet ports. These fiber bundles are potted or sealed at each end so that any liquid entering the ends of the cartridge will necessarily flow through the interior of the fibers. Cells may be seeded inside and/or outside the fibers within the cartridge in the extra capillary space (ECS). In some embodiments, the hollow fiber bioreactor comprises a single tube made up of a plurality of fibers (e.g., electrospun fibers). In some embodiments, the hollow fiber bioreactor comprises one or more tubes made up of a plurality of fibers.

Three fundamental characteristics differentiate hollow fiber cell culture from other methods: (1) cells are bound to a porous matrix much as they are in vivo, not a plastic dish (for example), (2) the molecular weight cut off of the support matrix can be controlled, and (3) extremely high surface area to volume ratio (150 cm² or more per mL) which provides a large area for metabolite and gas exchange for efficient growth of host cells.

The bioreactor structure includes a fiber matrix (e.g., three-dimensional scaffold as described herein) that allows permeation of nutrients, gases and other basic media components, as well as cell waste products, but not cells, where the cells can be amplified. The hollow fibers help to create a semi-permeable barrier between the cell growth chamber and the medium flow. Since the surface area provided by this design is large, using this fiber as a culture substrate allows the production of large numbers of cells. Cells growing in the 3-dimensional environment within the bioreactor are bathed in fresh medium as it perfuses through the hollow fibers.

In configuring the hollow fiber bioreactor, design considerations and parameters for the scaffold can be varied (as described herein), depending upon the goals associated with expansion of the cells.

### Cell Constructs

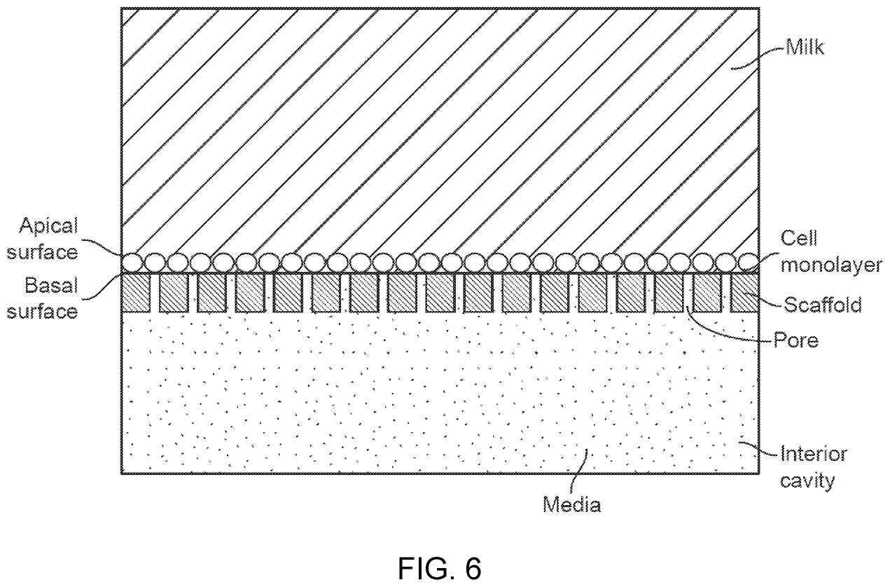

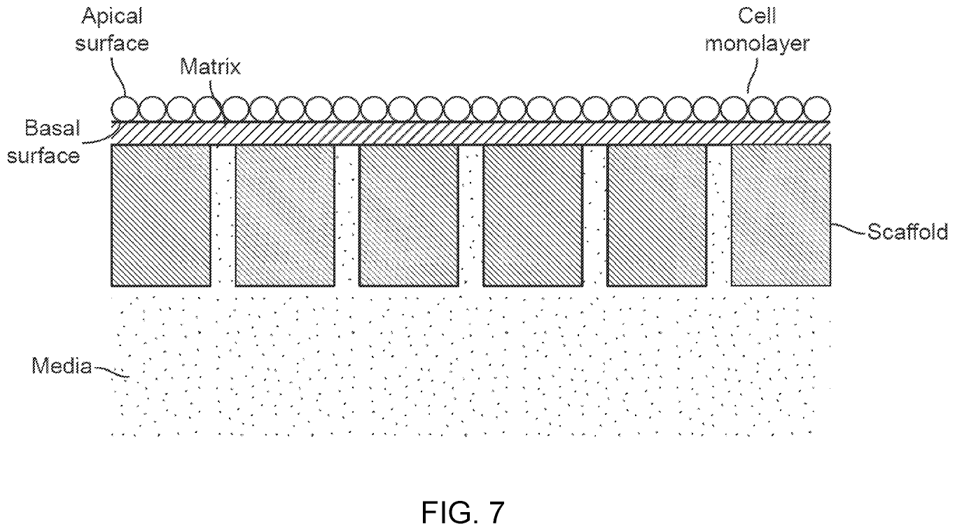

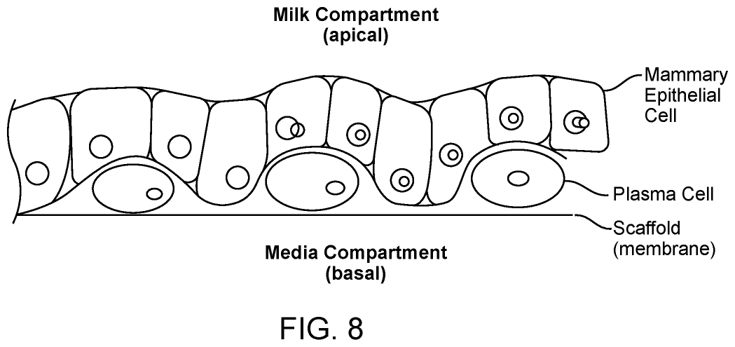

Described herein, in certain embodiments, are recombinant osteopontin and methods of producing recombinant osteopontin. In some embodiments, mammary epithelial cells (MECs) are used for producing recombinant osteopontin. In some embodiments, the cell constructs comprise a scaffold, a culture medium in fluidic contact with the scaffold, and mammary cells coupled to the scaffold. In some embodiments, the scaffold comprises a bottom surface/interior surface in fluid contact with the culture medium. In some embodiments, the scaffold comprises atop surface/exterior surface coupled to the MECs. In some embodiments, the MECs are coupled to the exterior surface in a continuous monolayer arrangement. In some embodiments, as described herein, the MECs are polarized and comprise an apical surface, and a basal surface, wherein the basal surface faces towards the exterior surface of the scaffold (see for example FIGS. 6-8).

In some embodiments, the cell constructs enable for compartmentalization between secreted milk from the mammary cells and the culture medium. In some embodiments, the lower surface (interior surface) of the scaffold is adjacent to a basal compartment. In some embodiments, the apical surface of the continuous monolayer (of the MECs) is adjacent to an apical compartment. In some embodiments, the continuous monolayer secretes milk through its apical surface into the apical compartment, thereby producing milk. In some embodiments, the monolayer of mammary cells forms a barrier that divides the apical compartment and the basal compartment, wherein the basal surface of the mammary cells is attached to the scaffold and the apical surface is oriented toward the apical compartment. In some embodiments, the milk product represents the biosynthetic output of cultured mammary epithelial cells (immortalized or from primary tissue samples) and immunoglobin A (IgA), immunoglobin G (IgG), and/or immunoglobin M (IgM) producing cells, for example plasma cells.

### Scaffolds

In some cases, features and/or properties of the scaffold are varied so as to help further the proliferation of mammary epithelial cells. For example, cellular microenvironment plays an important role in driving crucial cellular processes. In the context of mammary epithelial cells, the cellular microenvironment drives processes such as epithelial cell growth, epithelial differentiation and maintenance of epithelial phenotype, polarization, and production and secretion of milk components. The basement membrane (BM), which forms the physical boundary of the mammary gland and provides a support (or scaffolding) for the mammary epithelial cells can impact the development of the mammary gland through its influence on the mammary epithelial cell processes.

Generally, the basement membrane is a thin sheet that physically surrounds the mammary gland and can comprise of cross-linked fibrous networks (for example, comprising a plurality of nanofibers), such as Collagen-IV and laminins (predominantly laminin-1), along with other extracellular matrix (ECM) molecules, such as glycoproteins (like Nidogen) and proteoglycans. The basement membrane can serve as a semi-permeable scaffolding that allows for exchange of nutrients and waste metabolites to and from the mammary gland. Further, it also provides compartmentalization (barrier functionality) between secreted milk components and surrounding stroma and blood circulation. Moreover, the basement membrane can directly influence the ability of mammary epithelial cells to execute milk biosynthesis. For example, the basement membrane can provide mammary epithelial cells with i) bio-physical cues—through mechanical stimuli and its fibrous topographical features, and ii) bio-chemical cues—through its interactions with cells surface receptors called integrins. These bio-physical and bio-chemical cues together can influence the biology of mammary epithelial cells by regulating cell proliferation, epithelial differentiation, spatial organization of luminal and myoepithelial cells, polarization, alveologenesis and ductal morphogenesis, and activation of milk biosynthetic pathways and secretion. In some cases, the basement membrane is constantly being remodeled throughout the development, lactation, and involution of mammary glands to allow it to guide and control epithelial cell behavior. In the context of milk biosynthesis, in some cases, the basement membrane can regulate the Jak2-Stat5 pathway, and hence, prolactin signaling through its interactions with integrin receptors. Similarly, the basement membrane at other organ sites, such as kidney, cornea, and blood vessels, have been shown to have organ-specific topographical features. In certain instances, as a non-limiting example, culturing mammary epithelial cells in or on materials derived from a basement membrane associated in vivo with mammary cells or materials similar to materials derived from a basement membrane associated in vivo with mammary cells (including synthetic materials) promotes key functional aspects of such mammary cells, such as polarization and milk protein synthesis and secretion.

Described herein, in some embodiments, are scaffolds (as part of a cell construct, for example, configured to recapitulate one or more aspects of a basement membrane associated in vivo with mammary cells, and in some cases, the scaffolds are configured to induce the secretory phenotype of mammary epithelial cells in vitro. In some embodiments, such one or more aspects of a basement membrane include, for example, the fiber configuration (e.g., orientation of a plurality of fibers, such as nanofibers), porous nature, and/or other topographical features (e.g., mechanical stiffness and viscoelastic properties). In some embodiments, one or more properties and/or features of a scaffold are specified to at least partially mimic a basement membrane associated in vivo with mammary cells (e.g., a mammary gland). In some embodiments, the scaffold are produced with one or more synthetic materials and/or one or more natural materials (as described herein). In some embodiments, the scaffolds are produced in batch operation, continuous operations, or other processes known in the art for large scale production. In some cases, as a non-limiting example, specifying one or more properties and/or features facilitates batch-to-batch consistencies, scale-up and help reduce costs for large scale manufacturing of cell culturing platforms (in contrast with natural basement membrane derived materials which may pose challenges for such scale-up manufacturing and batch to batch consistencies).

### Optimization of Scaffold Features

In some embodiments, as described herein, the scaffold, as part of a cell construct described herein for example, includes a top surface/exterior surface and a bottom surface/interior surface. In some embodiments, the mammary cells are coupled to the top surface/exterior surface of the scaffold, and the bottom surface/interior surface of the scaffold is in fluid contact with the culture medium. In some embodiments, the scaffold comprises a 2-dimensional surface or a 3-dimensional surface (e.g., a 3-dimensional micropatterned surface, and/or as a cylindrical structure that is assembled into bundles). A non-limiting example of a 2-dimensional surface scaffold is a Transwell® filter.

In some embodiments, the scaffold comprises a three-dimensional surface. Non-limiting examples of a three-dimensional micropatterned surface include a microstructured bioreactor, a decellularized tissue (e.g., a decellularized mammary gland or decellularized plant tissue), micropatterned scaffolds fabricated through casting or three-dimensional printing with biological or biocompatible materials, textured surface.

In some embodiments, the scaffold is a three dimensional scaffold. In some embodiments, the scaffold comprises any shape, such as for example a sheet, sphere, mat, tubular structure or conduits. In some embodiments, the three dimensional scaffold comprises a tube structure or a flat sheet. For example, in some embodiments, the three-dimensional scaffold comprises any structure which has an enclosed hollow interior/central cavity. In some embodiments, the three-dimensional scaffold joins with one or more surfaces to form an enclosed interior chamber/basal compartment. For example, the scaffold can join with one or more walls of a bioreactor to form the interior chamber/basal compartment. In some embodiments, the scaffold is a hollow fiber bioreactor. In some embodiments, the three-dimensional scaffold is a tube in which the central cavity is defined by the interior surface of the scaffold. In some embodiments, the three-dimensional scaffold is a hollow sphere in which the central cavity is defined by the interior surface of the scaffold. In some embodiments, the scaffold comprises a mat configuration, which can be folded into a tube. In some embodiments, the tube has a diameter from about 0.1 mm to about 10 mm. In some embodiments, the tube has a diameter from about 0.5 mm to about 5 mm, from about 1 mm to about 3 mm, from about 1.5 mm to about 2.5 mm.

In some embodiments, a three-dimensional scaffold allows the cells (e.g., mammary epithelial cells and/or plasma cells) to grow or interact with their surroundings in all three dimensions. Unlike two-dimensional environments, in some cases, a three-dimensional cell culture allows cells in vitro to grow in all directions, thereby helping approximate the in vivo mammary environment. Further, the three-dimensional scaffold allows for a larger surface area for culture of the cells and for metabolite and gas exchange, plus it enables necessary compartmentalization—enabling the cultured milk product to be secreted into one compartment, while the cell culture media is contacted with the mammary cells and plasma cells via another compartment.

In some embodiments, the scaffold comprises a plurality of fibers (e.g., fibrous scaffold). In some embodiments, a population of the plurality of fibers are nanofibers (e.g., fibers having a diameter or thickness in the nanometer range, as described herein). In some embodiments, the plurality of fibers comprise one or more polymers (e.g., thermoplastic polyurethane, polycaprolactone, polyether sulfone (PES), polysulfone (PS), and/or polyvinylidene fluoride (PVDF)). In some embodiments, the one or more polymers (for example, of the fibers) comprise one or more polymer chains. In some cases, such materials recapitulate one or more bio-physical cues and/or one or more bio-chemical cues provided by the basement membrane. In some embodiments, the scaffold comprises a natural polymer, a biocompatible synthetic polymer, a synthetic peptide, and/or a composite derived from any combination thereof. In some embodiments, a natural polymer useful with this invention includes, but is not limited to, collagen, chitosan, cellulose, agarose, alginate, gelatin, elastin, heparan sulfate, chondroitin sulfate, keratan sulfate, and/or hyaluronic acid. In some embodiments, a biocompatible synthetic polymer useful with this invention includes, but is not limited to, cellulose, polysulfone, polyvinylidene fluoride, polyethylene co-vinyl acetate, polyvinyl alcohol, sodium polyacrylate, an acrylate polymer, polyethylene glycol, thermoplastic polyurethane (TPU), polycaprolactone (PCL), or a combination thereof. In some embodiments, the scaffold comprises TPU and/or PCL.

In some embodiment, the scaffold comprises a plurality of fibers that are oriented in a non-uniformly and/or non-linearly manner. For example, in some embodiments, the orientation for at least some of the plurality of fibers (e.g., from about 1% to about 99%) is a random orientation (thus non-uniform and/or non-linear with each other). For example in some embodiments, at least 1%, 5%, 10%, 20%, 25%, 33%, 50%, 66%, 75%, 80%, 90%, 99%, of the plurality of fibers in the scaffold are in a non-uniform and/or non-linear orientation (as compared with each other).

In some embodiments, the plurality of fibers form a fibrous/filamentous mesh. As described herein, in some embodiments, the plurality of fibers of the scaffold comprise nanofibers. In some embodiments, the fibrous scaffolds (e.g., scaffolds comprising a plurality of fibers, as described herein) are synthetic and can be formed via electrospinning, wet spinning, dry spinning, melt spinning, and/or phase inversion spinning of thermoplastic polyurethane and/or polycaprolactone. In some embodiments, the fibrous scaffolds can further be formed by electrospinning, wet spinning, dry spinning, melt spinning, and/or phase inversion spinning of other polymer material such as polyether sulfone (PES), polysulfone (PS), and/or polyvinylidene fluoride (PVDF). In some embodiments, such synthetic fibrous scaffolds (such as electrospun fibrous scaffolds) allow for tunability with respect to topographical properties and other mechanical properties, as well as surface chemistries. In some embodiments, the scaffold is produced by electrospinning cellulose nanofibers and/or a cylindrical structure that can be assembled into bundles (e.g., a hollow fiber bioreactor).

In some embodiments, the scaffold is at least partially permeable from the interior surface of the scaffold to exterior surface of the scaffold (and/or vice versa). In some embodiments, such permeability allows for fluid communication between the culture medium and the mammary cells coupled to the exterior surface of the scaffold. For example, in some embodiments, such permeability allows for i) the passage of nutrients to the cells, ii) waste to be carried away (e.g., from the cell layer to the culture medium (e.g., cell media), iii) provision of desired products to the cells (such as growth factors), iv) removal of desired products from the cells, v) exclusion of certain factors that may be present from reaching the cells, vi) other transfer of substances between the cell layer and culture media, or vii) any combination thereof.

In some embodiments, the scaffold is porous so as to enable such permeability between the interior surface and the exterior surface. In some embodiments, the scaffold comprises one or more pores (e.g., pores in the fiber walls of the scaffold) that may extend from the interior surface to the exterior surface. For example, in some embodiments, the pores are due to the fibrous configuration of the scaffold, such as due to the alignment and/or orientation of the plurality of fibers of the scaffold. Accordingly, in some embodiments, the one or more pores provides corresponding passageways through the plurality of fibers that allow the culture medium (cell media) to contact the cell layer coupled to the exterior surface of the scaffold (e.g., the basal surface of the cells of the cell monolayer of the MECs, as described herein). In some embodiments, the pore size of the fiber walls (of the scaffold) are specified so as to modify which components will pass through the walls.

In some embodiments, the pore size of a pore on the scaffold refers to a maximum dimension of a cross-section of a pore across the exterior surface of the scaffold. For example, if one of the pores comprises a circular cross-section as it traverses through the scaffold (e.g., from the exterior surface to the interior surface), the pore size refers to the diameter of the circular cross-section (in this case, the maximum dimension) at the exterior surface of the scaffold. In some embodiments, the pore size of a pore is substantially consistent with the maximum dimension of the pore as it traverses through the scaffold from the exterior surface to the interior surface. In some embodiments, the maximum dimension of the pore varies as it traverses through the scaffold from the exterior surface to the interior surface.

In some embodiments, the average diameter of the nanofiber is from about 100 nm to about 600 nm, from about 200 nm to about 500 nm, or from about 300 nm to about 400 nm. In some embodiments, the nanofiber is a flat sheet and has a fiber diameter from about 100 nm to about 600 nm. In some embodiments, the nanofiber is a tube and has a fiber diameter from about 100 nm to about 600 nm. In some embodiments, average fiber diameter for a PCL tube scaffold is higher than for a PCL flat sheet or a TPU flat sheet.

In some embodiments, the porosity of the scaffold is from about 5% to about 95%, from about 15% to about 75%, from about 25% to about 70%, or from about 40% to about 60%. In some embodiments, the porosity of the scaffold is from about 5% to about 95%, from about 15% to about 75%, from about 25% to about 70%, or from about 40% to about 60%. In some embodiments, the porosity of the nanofiber is from about 10% to about 35%, from about 15% to about 30%, or from about 20% to about 25%. In some embodiments, the nanofiber is a flat sheet and has a porosity from about 10% to about 35%. In some embodiments, the nanofiber is a tube and has a porosity from about 10% to about 35%.

In some embodiments, the scaffold has a specified density. In some embodiments, the plurality of pores have an average maximum dimension across the exterior surface from about 5 nm to about 1000 nm, from about 5 nm to about 50 nm, from about 50 nm to about 150 nm, from about 100 nm to about 500 nm, or from about 250 nm to about 1000 nm. In some embodiments, the plurality of pores have an average maximum dimension across the exterior surface from about 8 nm to about 10 nm, from about 25 nm to about 75 nm, from about 100 nm to about 250 nm, from about 200 nm to about 400 nm, or from about 300 nm to about 600 nm. In some embodiments, the plurality of pores have an average maximum dimension across the exterior surface that is less than or about the same as the average size (in diameter or length or as measured and/or sorted using a cell strainer giving rise to the average size definition for the cells) of the mammary cells. In some embodiments, the average size of the mammary cells is determined in a non-lactation stage of the cells.

In some embodiments, the average pore size of the scaffold is from about 1 nanometer² (nm²) to about 5 micrometer² (m²). In some embodiments, the average pore size of the scaffold is from about 1 nm² to about 20 nm². In some embodiments, the average pore size of the scaffold is from about 5 nm² to about 15 nm². In some embodiments, the average pore size of the scaffold is from about 8 nm² to about 10 nm². In some embodiments, the average pore size of the scaffold is at least about 5 nm². In some embodiments, the average pore size of the scaffold is at least about 9 nm². In some embodiments, the average pore size of the scaffold is at least about 25 nm². In some embodiments, the average pore size of the scaffold is at least about 50 nm². In some embodiments, the average pore size of the scaffold is at least about 100 nm². In some embodiments, the average pore size of the scaffold is at least about 0.5 μm². In some embodiments, the average pore size of the scaffold is at least about 1.0 μm². In some embodiments, the average pore size of the scaffold is at least about 1.5 μm². In some embodiments, the average pore size of the scaffold is at least about 2.0 μm². In some embodiments, the average pore size of the scaffold is at least about 2.5 μm². In some embodiments, the average pore size of the scaffold is at least about 3.0 μm².

In some embodiments, the average pore size of the nanofiber (measured as area, um²) is from about 5 nm² to about 600 nm², from about 100 nm² to about 500 nm², or from about 300 nm² to about 400 nm². In some embodiments, the nanofiber is a flat sheet and has a fiber pore size from about 5 nm² to about 600 nm². In some embodiments, the nanofiber is a tube and has a fiber pore size from about 100 nm² to about 600 nm². In some embodiments, the pore size for a PCL tube and TPU flat sheet is comparable.

In some embodiments, the average minimum Feret pore diameter of the nanofiber is from about 10 nm to about 600 nm, from about 200 nm to about 500 nm, or from about 300 nm to about 400 nm. In some embodiments, the nanofiber is a flat sheet and has a minimum Feret pore diameter from about 100 nm to about 600 nm. In some embodiments, the nanofiber is a tube and has a minimum Feret pore diameter from about 100 nm to about 600 nm.

In some embodiments, the average Maximum Feret pore diameter of the nanofiber is from about 30 nm to about 1300 nm, from about 200 nm to about 1200 nm, or from about 300 nm to about 1000 nm. In some embodiments, the nanofiber is a flat sheet and has a Maximum Feret pore diameter from about 300 nm to about 1200 nm. In some embodiments, the nanofiber is a tube and has a Maximum Feret pore diameter from about 100 nm to about 1300 nm.

In some embodiments, the average pore size of the scaffold is correlated with a size of protein passing through the scaffold. In some embodiments, the size of protein is correlated with the molecular weight of the protein. In some embodiments, the size of protein is measured in kilodalton (kDa) for example. Accordingly, in embodiments, the size of the protein (e.g., in kDa) that can pass through the pores is measured so as to determine an average pore size of the scaffold.

In some embodiments, the pore size is specified. As described herein, in some embodiments, the pore size is designed to allow the passage of nutrients to the cells, carry away waste, provide desired products to the cells (such as growth factors), to remove desired products from the cells, and/or exclude certain factors that may be present from reaching the cells.

Accordingly, the pore size of the fiber walls can be varied to modify which components will pass through the walls. For example, in some cases, pore size can allow the passage of large proteinaceous molecules, including growth factors, including, but not limited to, epidermal growth factor and platelet-derived growth factor. The person of ordinary skill in the art would understand how to vary the pore size depending upon the components that it is desirable to pass through the fiber walls to reach the cells or to carry material from the cells. As described herein, the pore size for both the scaffold (fiber walls) and/or the matrix material can be varied to allow for such transfer of materials between the cells and culture medium.

As described herein, in some embodiments, the scaffold is formed with one or more specified features configured to mimic that of a basement membrane (for example, a basement membrane associated in vivo with mammary cells). In some embodiments, the one or more specified features comprise one or more topological features, one or more mechanical properties, one or more surface properties, one or more viscoelastic properties, or a combination thereof.

In some embodiments, the one or more topological features of the scaffold are selected from i) an average fiber diameter of the plurality of fibers and ii) orientation(s) of the plurality of fibers. In some embodiments, as described herein, said average fiber diameter and/or orientation of the plurality of the fibers are varied and specified so as to configure the scaffold to at least partially mimic that of a basement membrane (for example, of a mammary gland).

In some embodiments, the average fiber diameter is from about 3 nm to about 10000 nm, from about 5 nm to about 5000 nm, from about 5 nm to about 50 nm, from about 50 nm to about 150 nm, from about 100 nm to about 300 nm, from about 100 nm to about 500 nm, from about 200 nm to about 1000 nm, from about 500 nm to about 1500 nm, from about 1000 nm to about 3000 nm, or from about 1500 nm to about 5000 nm. In some embodiments, the average diameter of the fibers of the plurality of fibers is characterized via SEM imaging.

As described herein, in some embodiments, the plurality of fibers are configured in a non-linear and/or non-uniform orientation. In some embodiments, the orientation of the plurality of fibers are randomly oriented with respect to each other. In some embodiments, the extent of fiber randomness is characterized using a scanning electron microscope (SEM) imaging through fast Fourier transform (FFT). For example, FFT may generate a point cloud from an image, wherein the proximity of points to each other indicates a similarity in orientation. Accordingly, a completely randomized SEM image may generate a homogenous point cloud (no discernable shape), whereas a more oriented sample may generate a skewed point cloud.

In some embodiments, the one or more mechanical properties of the scaffold are selected from: i) a thickness of the scaffold, ii) a modulus of elasticity of the scaffold (e.g., fibers), and iii) porosity (as described herein). In some embodiments, as described herein, said thickness of the scaffold, a modulus of elasticity of the scaffold (e.g., fibers), and/or porosity are varied and specified so as to configure the scaffold to at least partially mimic that of a basement membrane (for example, of a mammary gland).

In some embodiments, the thickness of the scaffold (e.g., comprising the plurality of fibers) is characterized through SEM imaging. In some embodiments, the thickness of the scaffold is from about 10 μm to about 500 μm. In some embodiments, the thickness of the scaffold is from about 15 μm to about 300 μm. In some embodiments, the thickness of the scaffold is from about 20 μm to about 200 μm. In some embodiments, the thickness of the scaffold is from about 20 μm to about 100 μm. In some embodiments, the thickness of the scaffold is from about 25 μm to about 75 μm. In some embodiments, the thickness of the scaffold is at least about 5 μm, 10 μm, 15 μm, or 20 μm. In some embodiments, the thickness of the scaffold is at most about 50 μm, 100 μm, 250 μm, 500 μm, or 1000 μm. In some embodiments, the average thickness of the scaffold is from about 40 nm to about 350 nm, from about 100 nm to about 300 nm, or from about 150 nm to about 200 nm. In some embodiments, the nanofiber is a flat sheet and has an average thickness of the scaffold from about 40 nm to about 150 nm. In some embodiments, the nanofiber is a tube and has an average thickness of the scaffold from about 100 nm to about 350 nm. In some embodiments, the average thickness of a PCL tube is higher than the average thickness of a PCL flat sheet or a TPU flat sheet.

In some embodiments, the modulus of elasticity is characterized through uniaxial tensile testing. In some embodiments, the scaffold comprises a modulus of elasticity from about 50 Pa to about 500 Pa. In some embodiments, the scaffold comprises a modulus of elasticity from about 100 Pa to about 300 Pa. In some embodiments, the scaffold comprises a modulus of elasticity from about 150 Pa to about 200 Pa. In some embodiments, one or more mechanical properties, or other topographical features of the scaffold is characterized using field emission scanning electron microscopy (FESEM).

In some embodiments, the one or more viscoelastic properties correlates to the entanglement of one or more fibers of the scaffold. As used here, “entanglement” means the interaction either i) of a polymer chain with itself (for example, similar to a single string having knots or tangled points with itself), or ii) between multiple polymer chains (for example, similar to multiple strings crossing over one another and forming one or more knots). In some embodiments, the one or more viscoelastic properties of the scaffold is controlled based on a specified ratio of a degree of entanglement of a polymer chain around itself (of a given nanofiber) to a degree of entanglement between two or more polymer chains (of the nanofibers).

In some embodiments, the porosity refers to i) a percent (%) porosity of the scaffold, ii) pore diameter or pore size (as described herein) through nitrogen porosimetry or mercury intrusion pore size analyzers such as Anton PaarMaster or MicroActive AutoPore V 9600, iii) a percent (%) range of porous area characterized through SEM imaging, and/or iv) a range of kD through dextran diffusion assay. In some embodiments, the porosity of the scaffold is correlated with the density of the scaffold, wherein a higher density (of the scaffold materials) correlates with a lower porosity. In some embodiments, the density of the scaffold is measured via a gas pycnometer.

In some embodiments, the one or more surface properties of the scaffold are selected from: i) the specific surface area, ii) hydrophobicity and/or hydrophilicity, iii) surface treatments to alters surface properties of the scaffold, iv) surface coatings, and v) an extent of surface coatings. In some embodiments, as described herein, said the specific surface area, hydrophobicity and/or hydrophilicity, surface treatments to alters surface properties of the scaffold, surface coatings, and/or an extent of surface coatings are varied and specified so as to configure the scaffold to at least partially mimic that of a basement membrane (for example, of a mammary gland).

The specific surface area can be characterized through the Brunauer Emmett Teller (BET) method or through SEM imaging. In some embodiments, the scaffold includes a specific area or region that is hydrophobic and/or a specific area or region that is hydrophilic. In some embodiments, an extent of hydrophobicity and/or hydrophilicity is measured via contact angle measurement. In some embodiments, the scaffold is subject to surface treatments, such as through plasma treatment, so as to alter hydrophobicity and/or hydrophilicity of the scaffold. In some embodiments the scaffold is subject to surface treatments such as poly-l-lysine coating to alter the surface charge (e.g., to make the surface more positively charged for cell attachment). In some embodiments the scaffold is subject to surface treatments such as coating with mussel inspired adhesive L-3,4-dihydroxyphenylalanine (L-DOPA) to alter the surface charge for enhanced cell attachment.

In some embodiments, a surface coating comprises extracellular matrix (ECM) and/or peptide coatings, as described herein for the matrix material (e.g., Collagen-IV, Laminin-1, RGD peptide, laminin peptides like IKVAV (SEQ ID NO: 6), other ECM-peptides). In some embodiments, an extent of a surface coating is varied, such as by specifying a concentration of coating solution, or through characterizing the total protein on the coated scaffold surface. In some embodiments, relative fluorescence units is used if using targeted staining methods for determining ECM coating on the scaffold surface.

### Mammary Cells

In some embodiments, the mammary cells (for example, for producing recombinant osteopontin) comprise milk-producing mammary epithelial cells (MECs), contractile myoepithelial cells, and/or progenitor cells that can give rise to both mammary epithelial cells (MECs) and mammary contractile myoepithelial cells. Mammary epithelial cells (MECs) are the only cells that produce milk. In some embodiments, the mammary cells comprise mammary epithelial cells (MECs), primary mammary epithelial cells, mammary myoepithelial cells and mammary progenitor cells. In some embodiments, the mammary cells are obtained from a tissue biopsy of a mammary gland.

In some embodiments, the mammary cells are derived from breast milk-derived stem cells or breast stem cells originating from tissue biopsy of a mammary gland. The epithelial component of breast milk includes not only mature epithelial cells, but also their precursors and stem cells in culture. A subpopulation of breast milk-derived stem cells displays very high multilineage potential, resembling those typical for human embryonic stem cells (hESCs). Breast stem cells may also originate from tissue biopsy of the mammary gland, and include terminally differentiated MECs. Both breast milk-derived stem cells and breast stem cells originating from tissue biopsy of the mammary gland are multi-potent cells that can give rise to MECs or myoepithelial cells.

In some embodiments, at least 50% of the mammary cells of the cells culture are polarized. In some embodiments, at least 55% of the mammary cells of the cell culture are polarized. In some embodiments, at least 60% of the mammary cells of the cell culture are polarized. In some embodiments, at least 65% of the mammary cells of the cell culture are polarized. In some embodiments, at least 70% of the mammary cells of the cell culture are polarized. In some embodiments, at least 75% of the mammary cells of the cell culture are polarized. In some embodiments, at least 80% of the mammary cells of the cell culture are polarized. In some embodiments, at least 85% of the mammary cells of the cell culture are polarized. In some embodiments, at least 90% of the mammary cells of the cell culture are polarized. In some embodiments, at least 95% of the mammary cells of the cell culture are polarized. In some embodiments, at least 100% of the mammary cells of the cell culture are polarized. In some embodiments, substantially all of the mammary cells of the cell construct are polarized (i.e., have an apical surface and a basal surface). In some embodiments, substantially all the mammary cells of the cell construct are polarized and substantially all the polarized cells are oriented in the same direction. For example, in some embodiments, substantially all of the mammary cells have an apical surface and a basal surface, wherein the apical surface of substantially all of the cells is oriented in the same direction and the basal surface of substantially all of the cells is oriented in the same direction.

In some embodiments, the continuous monolayer of mammary cells has at least 50% confluence over the scaffold. In some embodiments, the continuous monolayer of mammary cells has at least 60% confluence over the scaffold. In some embodiments, the continuous monolayer of mammary cells has at least 70% confluence over the scaffold. In some embodiments, the continuous monolayer of mammary cells has at least about 75% confluence over the scaffold. In some embodiments, the continuous monolayer of mammary cells has at least about 80% confluence over the scaffold. In some embodiments, the continuous monolayer of mammary cells has at least about 85% confluence over the scaffold. In some embodiments, the continuous monolayer of mammary cells has at least about 90% confluence over the scaffold. In some embodiments, the continuous monolayer of mammary cells has at least about 95% confluence over the scaffold. In some embodiments, the continuous monolayer of mammary cells has at least about 99% confluence over the scaffold. In some embodiments, the continuous monolayer of mammary cells has 100% confluence over the scaffold.

In some embodiments, the cell density of the continuous monolayer of mammary cells has at least 5.000 cells/cm² on the scaffold. In some embodiments, the cell density of the continuous monolayer of mammary cells has at least 10.000 cells/cm² on the scaffold. In some embodiments, the cell density of the continuous monolayer of mammary cells has at least 20.000 cells/cm² on the scaffold. In some embodiments, the cell density of the continuous monolayer of mammary cells has at least 30.000 cells/cm² on the scaffold. In some embodiments, the cell density of the continuous monolayer of mammary cells has at least 40.000 cells/cm² on the scaffold. In some embodiments, the cell density of the continuous monolayer of mammary cells has at least 50.000 cells/cm² on the scaffold. In some embodiments, the cell density of the continuous monolayer of mammary cells has at least 60.000 cells/cm² on the scaffold. In some embodiments, the cell density of the continuous monolayer of mammary cells has at least 70.000 cells/cm² on the scaffold. In some embodiments, the cell density of the continuous monolayer of mammary cells has at least 80.000 cells/cm² on the scaffold. In some embodiments, the cell density of the continuous monolayer of mammary cells has at least 90.000 cells/cm² on the scaffold. In some embodiments, the cell density of the continuous monolayer of mammary cells has at least 100.000 cells/cm² on the scaffold. In some embodiments, the cell density of the continuous monolayer of mammary cells has at least 150.000 cells/cm² on the scaffold. In some embodiments, the cell density of the continuous monolayer of mammary cells has at least 200.000 cells/cm² on the scaffold.

In some embodiments, the scaffold, or at least portion of the scaffold, is uncoated.

In some embodiments, the top surface/exterior surface of the scaffold is coated with a matrix material. In some embodiments, the matrix is made up of one or more extracellular matrix proteins. Non-limiting examples of extracellular matrix proteins include collagen, laminin, entactin, tenascin, and/or fibronectin. In some embodiments, the top of the scaffold is coated with Laminin-1, Collagen-IV, RGD peptide, laminin peptides like IKVAV (SEQ ID NO: 6), other ECM-peptides, or a combination thereof.

In some embodiments, the matrix material is located between the exterior surface of the scaffold and the mammary epithelial cells. In some embodiments, the matrix material is porous. In some embodiments, the matrix material is permeable to the cell media, allowing the cell media to contact the cells of the layer of the mammary cells. In some embodiments, the matrix material is transversed by at least one pore that allows the cell media to contact the layer(s) of mammary epithelial cells. In some embodiments, the matrix material comprises pores having an average pore size (as described herein, for example with reference to the scaffold pores) that corresponds with the average pore size of the scaffold (as described herein). In some embodiments, the pores of the matrix material are at least partially aligned with the pores of the scaffold. In some embodiments, the pores of the matrix material are randomly situated, and thereby may or may not be aligned with any of the pores the scaffold. In some embodiments, a ECM-coated PCL scaffold supports the self-organization of cells into distinct structures to a higher extend than uncoated PCL or ECM-coated TPU scaffold.

In some embodiments, the range of the average pore size (as described herein, for example with reference to the scaffold pores) of the pores in the matrix material is similar to the range in the average pore size of the pores for the scaffold, as described herein.

### Genetic Modifications to Mammary Cells

In some embodiments, the mammary cells comprise one or more genetic modification. For example, in some embodiments, the mammary cells comprise a constitutively active prolactin receptor protein. In some embodiments, the mammary cells comprise a constitutively active human prolactin receptor protein. Where the primary mammary epithelial cell or immortalized mammary epithelial cells comprise a constitutively active prolactin receptor, the culture medium does not contain prolactin.

In some embodiments, the constitutively active human prolactin receptor protein comprises a deletion of amino acids, as described in PCT Publication WO2021242866A1.

In some embodiments, the mammary cells comprise a loss of function mutation introduced into a circadian related gene PER2, as described in PCT Publication WO2021242866A1. In some embodiments, the loss of function mutation introduced into a circadian related gene PER2 promotes increased synthesis of cultured milk components.

In some embodiments, the mammary cells comprise a polynucleotide encoding a prolactin receptor comprising a modified intracellular signaling domain, as described in PCT Publication WO2021242866A1. In some embodiments, the loss of function mutation introduced into a circadian related gene PER2 promotes increased synthesis of individual cultured milk components.

In some embodiments, the mammary cells comprise a polynucleotide encoding a modified (e.g., recombinant) effector of a prolactin protein, as described in PCT Publication WO2021242866A1. In some embodiments, the modified effector of the prolactin protein comprises a janus kinase-2 (JAK2) tyrosine kinase domain. In some embodiments, the modified effector comprises a JAK2 tyrosine kinase domain fused to a signal transducer and activator of transcription-5 (STAT5) tyrosine kinase domain (e.g., a polynucleotide encoding a JAK2 tyrosine kinase domain linked to the 3′ end of a polynucleotide encoding the STAT5 tyrosine kinase domain). In some embodiments, the modified effector of a prolactin protein promotes increased synthesis of individual cultured milk components.

In some embodiments, the mammary cells are transduced with one or more lentiviral vectors. In some embodiments, the lentiviral vector comprises a selection marker. In some embodiments the selection marker is selected from the group of: ampicillin, puromycin, hygromycin, neomycin, or blasticidin. In some embodiments, the lentiviral vector comprises a nucleotide sequence encoding a short-hairpin RNA (shRNA). In some embodiments, expression of the shRNA is under control of the AOX1, CMV, CAG, GALL, GAL10, H1, PGK, polyhedrin, SV40, T7, Tac, U6, UBC or EF-1α promoter. In some embodiments, the shRNA is directed to p16INK4. In some embodiments, the shRNA directed to p161NK4 suppresses p16INK4 activity. In some embodiments, the lentiviral vector comprises a nucleotide sequence encoding the catalytic subunit of human telomerase reverse transcriptase (hTERT). In some embodiments, expression of the hTERT is under control of the AOX1, CMV, CAG, GAL1, GAL10, H1, PGK, polyhedrin, SV40, T7, Tac, U6, UBC or EF-1α promoter.

### Plasma Cells

Plasma cells are derived from a human donor. In some embodiments, the plasma cells are derived from bone marrow, spleen, and/or a lymph node. In certain embodiments, the plasma cells are derived from mucosal epithelial cells other than mammary cells (e.g., from oronasal, gastrointestinal, or respiratory tissue). In some embodiments, the plasma cells are derived from a plasma cell line. In certain embodiments, the plasma cells are derived from a plasmacyte cell line. In some embodiments, the plasma cells are isolated and sorted from non-plasma cells via fluorescence-activated cell sorting, magnetic-activated cell sorting, and/or microfluidic cell sorting. In some embodiments, plasma cells, plasmablasts, or pre-plasmablasts are sorted and isolated by FACS analysis using markers known in the art (e.g., CD38, CD138 and/or CD19). In certain embodiments, the plasma cells are cultivated with the immortalized mammary epithelial cells on a scaffold, thereby producing a cell construct for producing a cultured milk product with secretory products of the plasma cells and mammary cells (e.g., sIgA, IgG, and/or sIgM). In certain embodiments, the plasma cells are grown on a scaffold below a monolayer of mammary cells. In certain embodiments, the plasma cells are grown as dispersed populations of plasma cells overlayed by a monolayer of mammary cells. In certain embodiments, the plasma cells are stimulated to produce immunoglobins during co-culture with mammary cells. In certain embodiments, the plasma cells produce one or more immunoglobins of a class selected from IgG, IgM and IgA. In certain embodiments the plasma cells produce IgA and/or IgM. In certain embodiments, plasma cells produce IgA and/or IgM, and the IgA and/or IgM is processed by mammary epithelial cells to yield sIgA and/or sIgM that is bound to secretory component, and the sIgA and/or sIgM is secreted by the apical surface of the mammary cells.

### Methods of Making Cell Constructs

Described herein, in certain embodiments, are recombinant osteopontin and methods of producing recombinant osteopontin using a cell construct. In some embodiments, the method comprises (a) depositing isolated mammary epithelial cells, mammary myoepithelial cells and/or mammary progenitor cells on the upper surface (exterior surface) of a scaffold having an upper surface and lower surface; (b) cultivating the mammary cells of (a) on the scaffold, to produce a monolayer of polarized mammary cells located above the upper surface of the scaffold, wherein the upper surface is located adjacent to and above the lower surface of the scaffold, and wherein the polarized mammary cells comprise an apical surface and a basal surface, thereby producing a cell construct for producing the cultured milk product. In some embodiments, the mammary cells are primary mammary cells. In some embodiments, the mammary cells are immortalized. In some embodiments, the mammary cells are derived from a cell culture. In some embodiments, the mammary epithelial cells, myoepithelial cells and/or mammary progenitor cells are isolated from bone marrow, spleen tissue, lymph node tissue, mammary explants from mammary tissue (e.g., breast, udder, teat tissue), or raw breastmilk. In some embodiments, the mammary cells comprise mammary epithelial cells. In some embodiments, the mammary cells, comprise mammary myoepithelial cells. In some embodiments, the mammary cells, comprise mammary progenitor cells. In some embodiments, plasma cells are also deposited on the exterior surface of the scaffold, to produce a mixed population of plasma cells and mammary cells (i.e., mammary epithelial cells, mammary myoepithelial cells and/or mammary progenitor cells). In some embodiments, one or more properties and features of the scaffold is specified (as described herein) so as to help mimic a basement membrane. In some embodiments, the plasma cells are deposited onto the surface of the scaffold prior to the deposition of the mammary cells. In some embodiments, the plasma cells are isolated from any suitable human tissue or a cell culture.

In certain embodiments, the plasma cells are stimulated to produce immunoglobins during co-culture. In certain embodiments, the plasma cells produce one or more immunoglobins of a class selected from IgG, IgM and IgA. In certain embodiments the plasma cells produce secretory IgA. In certain embodiments, plasma cells are co-cultured with MECs in a bioreactor according to methods described herein. In certain embodiments, the bioreactor is a hollow fiber bioreactor described herein.

In certain embodiments, mammary cells are modified and/or stimulated with prolactin according to the methods described herein to stimulate and optimize milk production. In certain embodiments, the mammary cells are modified to express a constitutively active prolactin receptor protein.

In certain embodiments, mammary cells are identified and isolated from mammary tissue samples. In some embodiments, the mammary cells are isolated and sorted via fluorescence-activated cell sorting, magnetic-activated cell sorting, and/or microfluidic cell sorting. In certain embodiments, the mammary epithelial cell populations are sorted by FACS analysis using markers known in the art for identifying the cell populations. In certain embodiments, myoepithelial mammary cells and luminal epithelial mammary cells are isolated by FACS analysis. In certain embodiments, progenitor myoepithelial mammary cells and/or progenitor luminal epithelial mammary cells are isolated by FACS analysis. Any suitable method known in the art for sorting mammary epithelial cells (e.g., luminal epithelial cells), myoepithelial cells, progenitor cells, and immune cells can be used. For example, mammary cells can be sorted using CD24, EPCAM and/or CD49f, cell surface markers.

In some embodiments, plasma cells are identified and isolated from primary mucosal tissue (e.g., oronasal, gastrointestinal, respiratory or mammary). In some embodiments, plasma cells are identified and isolated from primary mammary tissue samples. In some embodiments, the plasma cells are isolated and sorted via fluorescence-activated cell sorting, magnetic-activated cell sorting, and/or microfluidic cell sorting. In certain embodiments, plasma cells are sorted and isolated by FACS analysis. In certain embodiments plasma cells, plasmablasts, or pre-plasmablasts are sorted and isolated by FACS analysis using markers known in the art (e.g., CD20, CD38, CD138 and/or CD19).

In some embodiments, the culturing and/or cultivating of the mammary cells and/or plasma cells for the cell construct is carried out at a temperature of about 35 °C to about 39 °C (e.g., a temperature of about 35 °C, 35.5 °C, 36 °C, 36.5 °C, 37 °C, 37.5 °C, 38 °C, 38.5 °C or about 39 °C, or any value or range therein, e.g., about 35 °C to about 38 °C, about 36 °C to about 39 °C, about 36.5 °C to about 39 °C, about 36.5 °C to about 37.5 °C, or about 36.5 °C to about 38 °C). In some embodiments, the culturing and/or cultivating is carried out at a temperature of about 37 °C. In some embodiments, the culturing and/or cultivating of the mammary cells and/or plasma cells for the cell construct is carried out at a temperature of about 27 °C to about 39 °C (e.g., a temperature of about 27 °C, 27.5 °C, 28 °C, 28.5 °C, 29 °C, 29.5 °C, 30 °C, 30.5 °C, 31 °C, 31.5 °C, 32 °C, 32.5 °C, 33 °C, 33.5 °C, 34 °C, 34.5 °C, 35 °C, 35.5 °C, 36 °C, 36.5 °C, 37 °C, 37.5 °C, 38 °C, 38.5 °C or about 39 °C, or any value or range therein, e.g., about 35 °C to about 38 °C, about 36 °C to about 39 °C, about 36.5 °C to about 39 °C, about 36.5 °C to about 37.5 °C, or about 36.5 °C to about 38 °C). In some embodiments, the culturing and/or cultivating of the mammary cells and/or plasma cells for the cell construct is carried out at a temperature of about 30 °C to about 39 °C (e.g., a temperature of about 30 °C, 30.5 °C, 31 °C, 31.5 °C, 32 °C, 32.5 °C, 33 °C, 33.5 °C, 34 °C, 34.5 °C, 35 °C, 35.5 °C, 36 °C, 36.5 °C, 37 °C, 37.5 °C, 38 °C, 38.5 °C or about 39 °C, or any value or range therein, e.g., about 35 °C to about 38 °C, about 36 °C to about 39 °C, about 36.5 °C to about 39 °C, about 36.5 °C to about 37.5 °C, or about 36.5 °C to about 38 °C).

In some embodiments, the culturing and/or cultivating of the mammary cells and/or plasma cells for the cell construct is carried out at an atmospheric concentration of CO2 of about 4% to about 6%, e.g., an atmospheric concentration of CO2 of about 4%, 4.25%, 4.5%, 4.75%, 5%, 5.25%, 5.5%, 5.75%, or 6% or any value or range therein, e.g., about 4% to about 5.5%, about 4.5% to about 6%, about 4.5% to about 5.5%, or about 5% to about 6%). In some embodiments, the culturing and/or cultivating is carried out at an atmospheric concentration of CO2 of about 5%.

In some embodiments, the culturing and/or cultivating of the mammary cells and/or the plasma cells for the cell construct comprises culturing and/or cultivating in a culture medium that is exchanged about every day to about every 10 days (e.g., every 1 day, every 2 days, every 3 days, every 4 days, every 5 days, every 6 days, every 7 days, every 8 days, every 9 days, every 10 days, or any value or range therein, e.g., about every day to every 3 days, about every 3 days to every 10 days, about every 2 days to every 5 days). In some embodiments, the culturing and/or cultivating further comprises culturing in a culture medium that is exchanged about every day to about every few hours to about every 10 days, e.g., about every 1, 2, 3, 4, 5, 6, 7, 8, 9, 10, 11, 12, 13, 14, 15, 16, 17, 18, 19, 20, 21, 22, 23, or 24 hours to about every 1, 2, 3, 4, 5, 6, 7, 8, 9, or 10 days or any value or range therein. For example, in some embodiments, the culturing and/or cultivating further comprises culturing and/or cultivating in a culture medium that is exchanged about every 12 hours to about every 10 days, about every 10 hours to about every 5 days, or about every 5 hours to about every 3 days.

In some embodiments, the cell construct is stored in a freezer or in liquid nitrogen. The storage temperature depends on the desired storage length. For example, freezer temperature (e.g., storage at a temperature of about 0 °C to about −80 °C or less, e.g., about 0 °C, −10 °C, −20 °C, −30 °C, −40 °C, −50 °C, −60 °C, −70 °C, −80 °C, −90 °C, −100 °C or any value or range therein) may be used if the cells are to be used within 6 months (e.g., within 1, 2, 3, 4, 5, or 6 months). For example, liquid nitrogen may be used (e.g., storage at a temperature of −100 °C or less (e.g., about −100 °C, −110 °C, −120 °C, −130, −140, −150, −160, −170, −180, −190 °C, −200 °C, or less) for longer term storage (e.g., storage of 6 months or longer, e.g., 6, 7, 8, 9, 10, 11, or 12 months, or 1, 2, 3, 4, 5, 6 or more years).

In some embodiments, the cell construct comprises a scaffold (as described herein) comprising an upper surface and a lower surface and a continuous monolayer of polarized mammary epithelial cells, a continuous monolayer of a polarized, mixed population of mammary epithelial cells, mammary myoepithelial cells and mammary progenitor cells, and/or a continuous monolayer of polarized immortalized mammary epithelial cells, wherein the continuous monolayer is located on the upper surface of scaffold. In some embodiments, the scaffold comprises a three dimensional scaffold (as described herein) comprising a plurality of fibers that are non-uniformly oriented and/or non-linearly oriented fibers. In some embodiments, the fibers comprise thermoplastic polyurethane and/or polycaprolactone. In some embodiments, the fibers comprise nanofibers.

In some embodiments, the lower surface of the scaffold is adjacent to the basal compartment. In some embodiments, the apical surface of the continuous monolayer is adjacent to the apical compartment. In some embodiments, the continuous monolayer secretes milk and sIgA or IgA through its apical surface into the apical compartment, thereby producing milk comprising IgA and/or sIgA in culture. In some embodiments, the continuous monolayer secretes milk and IgG through its apical surface into the apical compartment, thereby producing milk comprising IgG in culture. In some embodiments, the continuous monolayer secretes milk and sIgM or IgM through its apical surface into the apical compartment, thereby producing milk comprising IgM and/or sIgM in culture.

In some embodiments, the monolayer of mammary cells forms a barrier that divides the apical compartment and the basal compartment, wherein the basal surface of the mammary cells is attached to the scaffold and the apical surface is oriented toward the apical compartment.

In some embodiments, the basal compartment is adjacent to the lower surface of the scaffold. In some embodiments, the basal compartment comprises a culture medium in fluidic contact with the basal surface of the monolayer of mammary epithelial cells (e.g., the polarized monolayer of mammary epithelial cells, the polarized the monolayer of the mixed population of mammary cells, or the polarized monolayer of immortalized mammary epithelial cells).

In some embodiments, the culture medium comprises a carbon source, a chemical buffering system, one or more essential amino acids, one or more vitamins and/or cofactors, and one or more inorganic salts.

In some embodiments, the bioreactor comprises an apical compartment that is adjacent to the apical surface of the monolayer. In some embodiments, the apical compartment is adjacent to the upper surface of the scaffold.

In some embodiments, the bioreactor maintains a temperature of about 27 °C to about 39 °C (e.g., a temperature of about 27 °C, 28 °C, 29 °C, 30 °C, 31 °C, 32 °C, 33 °C, 34 °C, 35 °C, 35 °C, 35.5 °C, 36 °C, 36.5 °C, 37 °C, 37.5 °C, 38 °C, 38.5 °C or about 39 °C, or any value or range therein, e.g., about 27 °C to about 38 °C, about 36 °C to about 39 °C, about 36.5 °C to about 39 °C, about 36.5 °C to about 37.5 °C, or about 36.5 °C to about 38 °C). In some embodiments, the bioreactor maintains a temperature of about 37 °C.

In some embodiments, the bioreactor has an atmospheric concentration of CO2 of about 4% to about 6%, e.g., an atmospheric concentration of CO2 of about 4%, 4.25%, 4.5%, 4.75%, 5%, 5.25%, 5.5%, 5.75%, or 6% or any value or range therein, e.g., about 4% to about 5.5%, about 4.5% to about 6%, about 4.5% to about 5.5%, or about 5% to about 6%). In some embodiments, the bioreactor has an atmospheric concentration of CO2 of about 5%.

In some embodiments, the bioreactor has an atmospheric concentration of CO2 of about 4% to about 6%, e.g., an atmospheric concentration of CO2 of about 4%, 4.25%, 4.5%, 4.75%, 5%, 5.25%, 5.5%, 5.75%, or 6% or any value or range therein, e.g., about 4% to about 5.5%, about 4.5% to about 6%, about 4.5% to about 5.5%, or about 5% to about 6%). In some embodiments, the bioreactor has an atmospheric concentration of CO2 of about 5%.

In some embodiments, the method comprises monitoring the concentration of dissolved 02 and CO2. In some embodiments, the concentration of dissolved 02 is maintained between about 10% to about 25% or any value or range therein (e.g., about 10, 11, 12, 13, 14, 15, 16, 17, 18, 19, 20, 21, 22, 23, 24, or 25%). For example, in some embodiments, the concentration of dissolved 02 is maintained between about 12% to about 25%, about 15% to about 22%, about 10% to about 20%, about 15%, about 20%, or about 22%. In some embodiments, the concentration of CO2 is maintained between about 4% to about 6%, e.g., a concentration of CO2 of about 4%, 4.25%, 4.5%, 4.75%, 5%, 5.25%, 5.5%, 5.75%, or 6% or any value or range therein, e.g., about 4% to about 5.5%, about 4.5% to about 6%, about 4.5% to about 5.5%, or about 5% to about 6%). In some embodiments, the concentration of CO2 is maintained at about 5%.

In some embodiments, the culture medium is exchanged about every day to about every 10 days (e.g., every 1 day, every 2 days, every 3 days, every 4 days, every 5 days, every 6 days, every 7 days, every 8 days, every 9 days, every 10 days, or any value or range therein, e.g., about every day to every 3 days, about every 3 days to every 10 days, about every 2 days to every 5 days). In some embodiments, the culture medium is exchanged about every day to about every few hours to about every 10 days, e.g., about every 1, 2, 3, 4, 5, 6, 7, 8, 9, 10, 11, 12, 13, 14, 15, 16, 17, 18, 19, 20, 21, 22, 23, or 24 hours to about every 1, 2, 3, 4, 5, 6, 7, 8, 9, or 10 days or any value or range therein. For example, in some embodiments, the culture medium is exchanged about every 12 hours to about every 10 days, about every 10 hours to about every 5 days, or about every 5 hours to about every 3 days.

In some embodiments, the method comprises monitoring the glucose concentration and/or rate of glucose consumption in the culture medium and/or in the lactogenic culture medium. In some embodiments, the prolactin is added when the rate of glucose consumption in the culture medium is steady state.

In some embodiments, the method further comprises applying transepithelial electrical resistance (TEER) to measure the maintenance of the monolayer of epithelial cells. TEER measures a voltage difference between the fluids (e.g., media) in two compartments (e.g., between the apical and basal compartments), wherein if the barrier between the compartments loses integrity, the fluids in the two compartments may mix. When there is fluid mixing, the voltage difference will be reduced or eliminated; a voltage difference indicates that the barrier is intact. In some embodiments, upon detection of a loss of voltage by TEER, a scaffold (e.g., a Transwell® filter, a microstructured bioreactor, a decellularized tissue, a hollow fiber bioreactor, etc.) is reinoculated with additional cells and allowed time to reestablish a barrier (e.g., a monolayer) before resuming production of the cultured milk product (e.g., milk production). In some embodiments, the TEER (as measured in Ohms*cm²) is from about −80 Ohms*cm² to about 200 Ohms*cm². In some embodiments, the TEER is at least about 0 Ohms*cm². In some embodiments, the TEER is at least about 10 Ohms*cm². In some embodiments, the TEER is at least about 20 Ohms*cm². In some embodiments, the TEER is at least about 30 Ohms*cm². In some embodiments, the TEER is at least about 40 Ohms*cm². In some embodiments, the TEER is at least about 50 Ohms*cm². In some embodiments, the TEER is at least about 60 Ohms*cm². In some embodiments, the TEER is at least about 70 Ohms*cm². In some embodiments, the TEER is at least about 80 Ohms*cm². In some embodiments, the TEER is at least about 90 Ohms*cm². In some embodiments, the TEER is at least about 100 Ohms*cm². In some embodiments, the TEER is at least about 150 Ohms*cm². In some embodiments, the TEER is at least about 200 Ohms*cm². In some embodiments, the TEER increases with the duration of cell culture. In some embodiments, a scaffold with extra cellular matrix (ECM)-coated TPU has a higher average TEER value than a scaffold with ECM-coated PCL, ECM-coated PET, uncoated TPU, uncoated PCL, or uncoated PET.

In some embodiments, the method further comprises collecting the cultured milk product from the apical compartment to produce collected cultured milk product. In some embodiments, the collecting is via a port, via gravity, and/or via a vacuum. In some embodiments, a vacuum is attached to a port.

### Basal Culture Media and Lactogenic Media

In some embodiments, the culture medium comprises a carbon source, a chemical buffering system, one or more essential amino acids, one or more vitamins and/or cofactors, and one or more inorganic salts. In some embodiments, the carbon source, chemical buffering system, one or more essential amino acids, one or more vitamins and/or cofactors, and/or one or more inorganic salts are food grade. As used herein, the term “culture medium”, “culture media”, “cell medium”, and/or “cell media” may be used interchangeably.

In some embodiments, the culture medium is lactogenic culture medium. In some embodiments, the culture medium further comprises prolactin (e.g., mammalian prolactin, e.g., human prolactin), linoleic and alpha-linoleic acid, estrogen and/or progesterone. For example, in some embodiments, the culture medium comprises prolactin (or prolactin is added) in an amount from about 20 ng/mL to about 200 ng/L of culture medium, e.g., about 20, 30, 40, 50, 60, 70, 80, 90, 100, 110, 120, 130, 140, 150, 160, 170, 180, 190, or 200 ng/mL or any value or range therein. In some embodiments, the culture medium comprises prolactin (or prolactin is added) in an amount from about 20 ng/mL to about 195 ng/mL, about 50 ng/mL to about 150 ng/mL, about 25 ng/mL to about 175 ng/mL, about 45 ng/mL to about 200 ng/mL, or about 75 ng/mL to about 190 ng/mL of culture medium. In some embodiments, the culture medium further comprises other factors to improve efficiency, including, but not limited to, insulin, an epidermal growth factor, and/or a hydrocortisone.

In some embodiments, the culture medium comprises a carbon source in an amount from about 1 g/L to about 15 g/L of culture medium (e.g., about 1, 2, 3, 4, 5, 6, 7, 8, 9, 10, 11, 12, 13, 14 or 15 g/L or any value or range therein), or about 1, 2, 3, 4, 5 or 6 g/L to about 7, 8, 9, or 10, 11, 12, 13, 14 or 15 g/L of the culture medium. Non-limiting examples of a carbon source include glucose and/or pyruvate. For example, in some embodiments, the culture medium comprises glucose in an amount from about Ig/L to about 12 g/L of culture medium, e.g., about 1, 2, 3, 4, 5, 6, 7, 8, 9, 10, 11, or 12 g/L or any value or range therein. In some embodiments, the culture medium comprises glucose in an amount from about 1 g/L to about 6 g/L, about 4 g/L to about 12 g/L, about 2.5 g/L to about 10.5 g/L, about 1.5 g/L to about 11.5 g/L, or about 2 g/L to about 10 g/L of culture medium. In some embodiments, the culture medium comprises glucose in an amount from about 1, 2, 3, or 4 g/L to about 5, 6, 7, 8, 9, 10, 11, or 12 g/L or about 1, 2, 3, 4, 5, or 6 g/L to about 7, 8, 9, 10, 11, or 12 g/L. In some embodiments, the culture medium comprises pyruvate in an amount from about 5 g/L to about 15 g/L of culture medium, e.g., about 5, 6, 7, 8, 9, 10, 11, 12, 13, 14, or 15 g/L or any value or range therein. In some embodiments, the culture medium comprises pyruvate in an amount from about 5 g/L to about 14.5 g/L, about 10 g/L to about 15 g/L, about 7.5 g/L to about 10.5 g/L, about 5.5 g/L to about 14.5 g/L, or about 8 g/L to about 10 g/L of culture medium. In some embodiments, the culture medium comprises pyruvate in an amount from about 5, 6, 7, or 8 g/L to about 9, 10, 11, 12, 13, 14 or 15 g/L or about 5, 6, 7, 8, 9, or 10 g/L to about 11, 12, 13, 14 or 15 g/L.

In some embodiments, the culture medium comprises a chemical buffering system in an amount from about 1 g/L to about 4 g/L (e.g., about 1, 1.5, 2, 2.5, 3, 3.5, or 4 g/L or any value or range therein) of culture medium or about 10 mM to about 25 mM (e.g., about 10, 11, 12, 13, 14, 15, 16, 17, 18, 19, 20, 21, 22, 23, 24, or 25 mM or any value or range therein). In some embodiments, the chemical buffering system includes, but is not limited to, sodium bicarbonate and/or 4-(2-hydroxyethyl)-1-piperazineethanesulfonic acid (HEPES). For example, in some embodiments, the culture medium comprises sodium bicarbonate in an amount from about 1 g/L to about 4 g/L of culture medium, e.g., about 1, 1.5, 2, 2.5, 3, 3.5, or 4 g/L or any value or range therein. In some embodiments, the culture medium comprises sodium bicarbonate in an amount from about 1 g/L to about 3.75 g/L, about 1.25 g/L to about 4 g/L, about 2.5 g/L to about 3 g/L, about 1.5 g/L to about 4 g/L, or about 2 g/L to about 3.5 g/L of culture medium. In some embodiments, the culture medium comprises HEPES in an amount from about 10 mM to about 25 mM, e.g., about 10, 11, 12, 13, 14, 15, 16, 17, 18, 19, 20, 21, 22, 23, 24, or 25 mM or any value or range therein. In some embodiments, the culture medium comprises HEPES in an amount from about 11 mM to about 25 mM, about 10 mM to about 20 mM, about 12.5 mM to about 22.5 mM, about 15 mM to about 20.75 mM, or about 10 mM to about 20 mM.

In some embodiments, the culture medium comprises one or more essential amino acids in an amount from about 0.5 mM to about 5 mM (e.g., about 0.5, 1, 1.5, 2, 2.5, 3, 3.5, 4, 4.5, or 5 mM or any value or range therein) or about 0.5, 1, 1.5, 2 mM to about 2.5, 3, 3.5, 4, 4.5, or 5 mM. In some embodiments, the one or more essential amino acids is histidine, isoleucine, leucine, lysine, methionine, phenylalanine, threonine, tryptophan, valine, and/or arginine. For example, in some embodiments, the culture medium comprises arginine in an amount from about 0.5 mM to about 5 mM, e.g., about 0.5, 1, 1.5, 2, 2.5, 3, 3.5, 4, 4.5, or 5 mM or any value or range therein. In some embodiments, the culture medium comprises an essential amino acids in an amount from about 0.5 mM to about 4.75 mM, about 2 mM to about 3.5 mM, about 0.5 mM to about 3.5 mM, about 1 mM to about 5 mM, or about 3.5 mM to about 5 mM.

In some embodiments, the culture medium comprises one or more vitamins and/or cofactors in an amount from about 0.01 μM to about 50 μM (e.g., about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 3, 4, 5, 6, 7, 8, 9, 10, 12.5, 15, 17.5, 20, 25, 30, 35, 40, 45, 46, 47, 48, 49, 49.025, 49.05, 49.075, or 50 μM or any value or range therein) or about 0.01, 0.02, 0.03, 0.04, 0.05, 0.06, 0.07, 0.08, 0.09, 0.1, 0.2, 0.3, 0.4, 0.5, 0.6, 0.7, 0.8, or 0.9 μM to about 1, 1.1, 1.2, 1.3, 1.4, 1.5, 1.6, 1.7, 1.8, 1.9, 2, 2.1, 2.2, 2.3, 2.4, 2.5, 3, 4, 5, 6 μM or about 0.02, 0.025, 0.05, 0.075, 1, 1.5, 2, 3, 4, 5, 6, 7, 8, 9, 10 μM to about 12.5, 15, 17.5, 20, 25, 30, 35, 40, 45, 46, 47, 48, 49, 49.025, 49.05, 49.075, or 50 μM. In some embodiments, one or more vitamins and/or cofactors include, but are not limited to, thiamine and/or riboflavin. For example, in some embodiments, the culture medium comprises thiamine in an amount from about 0.025 μM to about 50 μM, e.g., about 0.025, 0.05, 0.075, 1, 1.5, 2, 3, 4, 5, 6, 7, 8, 9, 10, 12.5, 15, 17.5, 20, 25, 30, 35, 40, 45, 46, 47, 48, 49, 49.025, 49.05, 49.075, or 50 μM or any value or range therein. In some embodiments, the culture medium comprises thiamine in an amount from about 0.025 μM to about 45.075 μM, about 1 μM to about 40 μM, about 5 μM to about 35.075 μM, about 10 μM to about 50 μM, or about 0.05 μM to about 45.5 μM. In some embodiments, the culture medium comprises riboflavin in an amount from about 0.01 μM to about 3 μM, e.g., about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 μM or any value or range therein. In some embodiments, the culture medium comprises riboflavin in an amount from about 0.01 μM to about 2.05 μM, about 1 μM to about 2.95 μM, about 0.05 μM to about 3 μM, about 0.08 μM to about 1.55 μM, or about 0.05 μM to about 2.9 μM.

In some embodiments, the culture medium comprises one or more inorganic salts in an amount from about 100 mg/L to about 150 mg/L of culture medium (e.g., about 100, 105, 110, 115, 120, 125, 130, 135, 140, 145, or 150 mg/L or any value or range therein) or about 100 mg/L to about 150 mg/L of culture medium (e.g., about 100, 105, 110, 115, 120, 125, 130, 135, 140, 145, or 150 mg/L or any value or range therein). In some embodiments, one or more inorganic salts include, but are not limited to, calcium and/or magnesium. For example, in some embodiments, the culture medium comprises calcium in an amount from about 100 mg/L to about 150 mg/L of culture medium, e.g., about 100, 105, 110, 115, 120, 125, 130, 135, 140, 145, or 150 mg/L or any value or range therein. In some embodiments, the culture medium comprises arginine in an amount from about 100 mg/L to about 125 mg/L, about 105 mg/L to about 150 mg/L, about 120 mg/L to about 130 mg/L, or about 100 mg/L to about 145 mg/L of culture medium. In some embodiments, the culture medium comprises magnesium in an amount from about 0.01 mM to about 1 mM, e.g., about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 0.91, 0.92, 0.93, 0.94, 0.95, 0.96, 0.97, 0.98, 0.99, or 1 mM or any value or range therein. In some embodiments, the culture medium comprises magnesium in an amount from about 0.05 mM to about 1 mM, about 0.01 mM to about 0.78 mM, about 0.5 mM to about 1 mM, about 0.03 mM to about 0.75 mM, or about 0.25 mM to about 0.95 mM.

In some embodiments, the culture medium comprises a carbon source in an amount from about 1 g/L to about 15 g/L of culture medium (e.g., about 1, 2, 3, 4, 5, 6, 7, 8, 9, 10, 11, 12, 13, 14 or 15 g/L or any value or range therein), or about 1, 2, 3, 4, 5 or 6 g/L to about 7, 8, 9, or 10, 11, 12, 13, 14 or 15 g/L of the culture medium. In some embodiments, the carbon source includes, but is not limited to, glucose and/or pyruvate. For example, in some embodiments, the culture medium comprises glucose in an amount from about 1 g/L to about 12 g/L of culture medium, e.g., about 1, 2, 3, 4, 5, 6, 7, 8, 9, 10, 11, or 12 g/L or any value or range therein. In some embodiments, the culture medium comprises glucose in an amount from about 1 g/L to about 6 g/L, about 4 g/L to about 12 g/L, about 2.5 g/L to about 10.5 g/L, about 1.5 g/L to about 11.5 g/L, or about 2 g/L to about 10 g/L of culture medium. In some embodiments, the culture medium comprises pyruvate at an amount of about 5 g/L to about 15 g/L of culture medium, e.g., about 5, 6, 7, 8, 9, 10, 11, 12, 13, 14, or 15 g/L or any value or range therein. In some embodiments, the culture medium comprises pyruvate in an amount from about 5 g/L to about 14.5 g/L, about 10 g/L to about 15 g/L, about 7.5 g/L to about 10.5 g/L, about 5.5 g/L to about 14.5 g/L, or about 8 g/L to about 10 g/L of culture medium.

In some embodiments, the culture medium comprises a chemical buffering system in an amount from about 1 g/L to about 4 g/L (e.g., about 1, 1.5, 2, 2.5, 3, 3.5, or 4 g/L or any value or range therein) of culture medium or about 10 mM to about 25 mM (e.g., about 10, 11, 12, 13, 14, 15, 16, 17, 18, 19, 20, 21, 22, 23, 24, or 25 mM or any value or range therein). In some embodiments, the chemical buffering system includes, but is not limited to, sodium bicarbonate and/or HEPES. For example, in some embodiments, the culture medium comprises sodium bicarbonate in an amount from about 1 g/L to about 4 g/L of culture medium, e.g., about 1, 1.5, 2, 2.5, 3, 3.5, or 4 g/L or any value or range therein. In some embodiments, the culture medium comprises sodium bicarbonate in an amount from about 1 g/L to about 3.75 g/L, about 1.25 g/L to about 4 g/L, about 2.5 g/L to about 3 g/L, about 1.5 g/L to about 4 g/L, or about 2 g/L to about 3.5 g/L of culture medium. In some embodiments, the culture medium comprises HEPES in an amount from about 10 mM to about 25 mM, e.g., about 10, 11, 12, 13, 14, 15, 16, 17, 18, 19, 20, 21, 22, 23, 24, or 25 mM or any value or range therein. In some embodiments, the culture medium comprises HEPES in an amount from about 1 mM to about 25 mM, about 10 mM to about 20 mM, about 12.5 mM to about 22.5 mM, about 15 mM to about 20.75 mM, or about 10 mM to about 20 mM.

In some embodiments, the culture medium comprises one or more essential amino acids in an amount from about 0.5 mM to about 5 mM (e.g., about 0.5, 1, 1.5, 2, 2.5, 3, 3.5, 4, 4.5, or 5 mM or any value or range therein) or about 0.5, 1, 1.5, 2 mM to about 2.5, 3, 3.5, 4, 4.5, or 5 mM. In some embodiments, one or more essential amino acids is arginine and/or cysteine. For example, in some embodiments, the culture medium comprises arginine in an amount from about 0.5 mM to about 5 mM, e.g., about 0.5, 1, 1.5, 2, 2.5, 3, 3.5, 4, 4.5, or 5 mM or any value or range therein. In some embodiments, the culture medium comprises arginine in an amount from about 0.5 mM to about 4.75 mM, about 2 mM to about 3.5 mM, about 0.5 mM to about 3.5 mM, about 1 mM to about 5 mM, or about 3.5 mM to about 5 mM. For example, in some embodiments, the culture medium comprises cysteine in an amount from about 0.5 mM to about 5 mM, e.g., about 0.5, 1, 1.5, 2, 2.5, 3, 3.5, 4, 4.5, or 5 mM or any value or range therein. In some embodiments, the culture medium comprises cysteine in an amount from about 0.5 mM to about 4.75 mM, about 2 mM to about 3.5 mM, about 0.5 mM to about 3.5 mM, about 1 mM to about 5 mM, or about 3.5 mM to about 5 mM.

In some embodiments, the culture medium comprises one or more vitamins and/or cofactors in an amount from about 0.01 μM to about 50 μM (e.g., about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 3, 4, 5, 6, 7, 8, 9, 10, 12.5, 15, 17.5, 20, 25, 30, 35, 40, 45, 46, 47, 48, 49, 49.025, 49.05, 49.075, or 50 μM or any value or range therein) or about 0.01, 0.02, 0.03, 0.04, 0.05, 0.06, 0.07, 0.08, 0.09, 0.1, 0.2, 0.3, 0.4, 0.5, 0.6, 0.7, 0.8, or 0.9 μM to about 1, 1.1, 1.2, 1.3, 1.4, 1.5, 1.6, 1.7, 1.8, 1.9, 2, 2.1, 2.2, 2.3, 2.4, 2.5, 3, 4, 5, 6 μM or about 0.02, 0.025, 0.05, 0.075, 1, 1.5, 2, 3, 4, 5, 6, 7, 8, 9, 10 μM to about 12.5, 15, 17.5, 20, 25, 30, 35, 40, 45, 46, 47, 48, 49, 49.025, 49.05, 49.075, or 50 μM. In some embodiments, one or more vitamins and/or cofactors includes, but is not limited to, thiamine and/or riboflavin. For example, in some embodiments, the culture medium comprises thiamine in an amount from about 0.025 μM to about 50 μM, e.g., 0.025, 0.05, 0.075, 1, 1.5, 2, 3, 4, 5, 6, 7, 8, 9, 10, 12.5, 15, 17.5, 20, 25, 30, 35, 40, 45, 46, 47, 48, 49, 49.025, 49.05, 49.075, or 50 μM or any value or range therein. In some embodiments, the culture medium comprises thiamine in an amount from about 0.025 μM to about 45.075 μM, about 1 μM to about 40 μM, about 5 μM to about 35.075 μM, about 10 μM to about 50 μM, or about 0.05 μM to about 45.5 μM. In some embodiments, the culture medium comprises riboflavin in an amount from about 0.01 μM to about 3 μM, e.g., 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 μM or any value or range therein. In some embodiments, the culture medium comprises riboflavin in an amount from about 0.01 μM to about 2.05 μM, about 1 μM to about 2.95 μM, about 0.05 μM to about 3 μM, about 0.08 μM to about 1.55 μM, or about 0.05 μM to about 2.9 μM.

In some embodiments, the culture medium comprises one or more inorganic salts in an amount from about 100 mg/L to about 150 mg/L of culture medium (e.g., about 100, 105, 110, 115, 120, 125, 130, 135, 140, 145, or 150 mg/L or any value or range therein) or about 100 mg/L to about 150 mg/L of culture medium (e.g., about 100, 105, 110, 115, 120, 125, 130, 135, 140, 145, or 150 mg/L or any value or range therein). In some embodiments, exemplary one or more inorganic salts is calcium and/or magnesium. For example, in some embodiments, the culture medium comprises calcium in an amount from about 100 mg/L to about 150 mg/L of culture medium, e.g., about 100, 105, 110, 115, 120, 125, 130, 135, 140, 145, or 150 mg/L or any value or range therein. In some embodiments, the culture medium comprises arginine in an amount from about 100 mg/L to about 125 mg/L, about 105 mg/L to about 150 mg/L, about 120 mg/L to about 130 mg/L, or about 100 mg/L to about 145 mg/L of culture medium. In some embodiments, the culture medium comprises magnesium in an amount from about 0.01 mM to about 1 mM, e.g., about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 0.91, 0.92, 0.93, 0.94, 0.95, 0.96, 0.97, 0.98, 0.99, or 1 mM or any value or range therein. In some embodiments, the culture medium comprises magnesium in an amount from about 0.05 mM to about 1 mM, about 0.01 mM to about 0.78 mM, about 0.5 mM to about 1 mM, about 0.03 mM to about 0.75 mM, or about 0.25 mM to about 0.95 mM.

In some embodiments, the carbon source, chemical buffering system, one or more essential amino acids, one or more vitamins and/or cofactors, and/or one or more inorganic salts is food grade.

In some embodiments, the culture medium is lactogenic culture medium, e.g., the culture medium further comprises prolactin (e.g., mammalian prolactin, e.g., human prolactin). For example, in some embodiments, the culture medium comprises prolactin (or prolactin is added) in an amount from about 20 ng/mL to about 200 ng/L of culture medium, e.g., about 20, 30, 40, 50, 60, 70, 80, 90, 100, 110, 120, 130, 140, 150, 160, 170, 180, 190, or 200 ng/mL or any value or range therein. In some embodiments, the culture medium comprises prolactin (or prolactin is added) in an amount from about 20 ng/mL to about 195 ng/mL, about 50 ng/mL to about 150 ng/mL, about 25 ng/mL to about 175 ng/mL, about 45 ng/mL to about 200 ng/mL, or about 75 ng/mL to about 190 ng/mL of culture medium. In some embodiments, the methods further comprise adding prolactin to the culture medium, thereby providing a lactogenic culture medium. In some embodiments, the prolactin is produced by a microbial cell and/or a human cell expressing a recombinant prolactin (e.g., a prolactin comprising a substitution of a serine residue at position 179 of the prolactin gene with aspartate (S179D), e.g., S179D-prolactin). In some embodiments, adding prolactin to the culture medium comprises conditioning culture medium by culturing cells that express and secrete prolactin, and applying the conditioned culture medium comprising prolactin to the basal surface of the monolayer of mammary cells (e.g., mammary epithelial cells, mammary myoepithelial cells and mammary progenitor cells).

In some embodiments, the culture medium further comprises other factors to improve efficiency, including, but not limited to, insulin, an epidermal growth factor, and/or a hydrocortisone. In some embodiments, the methods of the present invention further comprise adding other factors (e.g., insulin, an epidermal growth factor, and/or a hydrocortisone) to the culture medium, e.g., to improve efficiency.

Having described the present disclosure, the same will be explained in greater detail in the following examples, which are included herein for illustration purposes only, and which are not intended to be limiting to the disclosure.

### EXAMPLES

The Examples that follow are illustrative of specific embodiments of the disclosure, and various uses thereof. They are set forth for explanatory purposes only and should not be construed as limiting the scope of the disclosure in any way.

### Example 1: Immortalization of Human Mammary Epithelial Cells

This example describes immortalization of human mammary epithelial cell (hMEC) lines via genomic modifications that directly target two primary senescence barriers encountered by cultured hMEC.

Human mammary epithelial cell (hMEC) lines were genetically modified using lentiviral vectors, and immortalization was achieved through suppression of p₁₆^{INK4} activity, and overexpression of human telomerase reverse transcriptase (hTERT). Specifically, hMEC were transduced with lentivirus expressing short-hairpin RNA (shRNA) directed to p₁₆^{INK4} hMEC were then transduced with lentivirus expressing the catalytic subunit of human telomerase reverse transcriptase (hTERT). Cell lines with the desired genomic modifications were identified via antibiotic selection.

### Example 2: Endogenous OPN Secretion in Immortalized Human Mammary Epithelial Cells

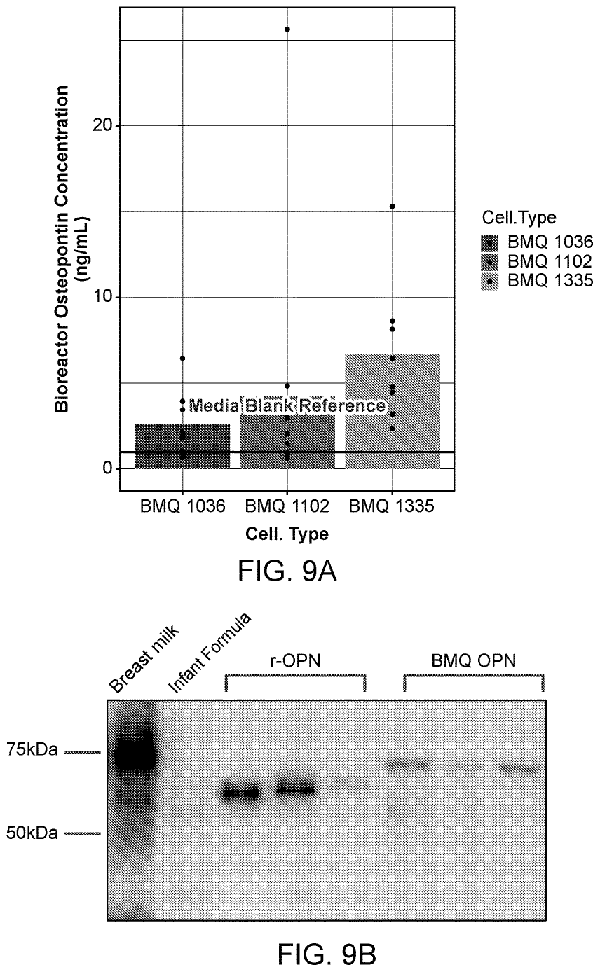

The following example shows that immortalized human mammary epithelial cells (hMECs) cultured in hollow fiber bioreactor (HFBR) format produce OPN (FIGS. 9A-9B)

Immortalized hMEC lines (BMQ 1036, BMQ 1102, and BMQ 1335) derived from three distinct samples of donor milk were expanded and seeded within hollow fiber bioreactors (HFBRs) at 40k cells/cm² and cultured for 4 weeks with media flowing through the intracapillary space (ICS) and samples being collected from the extracapillary space (ECS). Extracellular space was flushed and product was collected every 4 days. Average OPN was measured by ELISA (FIG. 9A). Western blot analysis was performed using antibody recognizing human OPN (FIG. 9B). Breast milk sample was diluted 1:50 in PBS. Infant formula sample consisted of an undiluted mixture of 3 different bovine based infant formulas. Recombinant OPN (r-OPN) samples consisted of: Hek293-derived human OPN; NSO-derived human OPN; and Bovine milk-derived OPN. BMQ OPN samples consisted of OPN generated from HFBR in BMQ1127 cells; OPN generated from HFBR in BMQ1127 cells treated with prolactin; and OPN generated from HFBR in BMQ1036 cells.

The data show that immortalized hMECs cultured in a hollow fiber bioreactor format produce OPN, and further demonstrate that OPN generated by immortalized hMEC is closer in size to breast milk OPN than to recombinant OPN or bovine OPN.

### Example 3: OPN Overexpression in Immortalized Human Mammary Epithelial

The following example describes the genetic modification of immortalized human mammary epithelial cell (hMEC) lines by lentiviral transduction to produce hMEC that overexpress OPN.

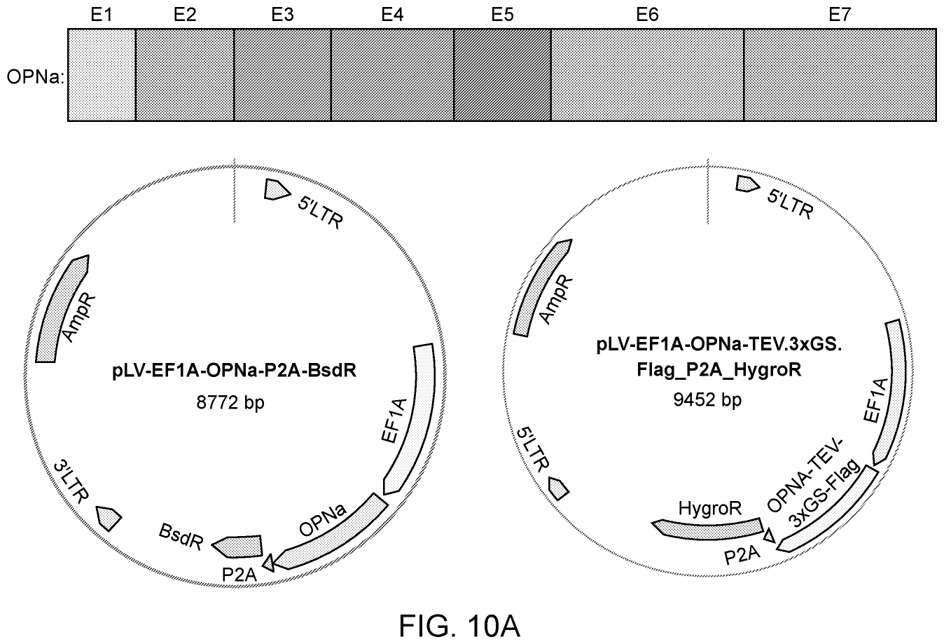

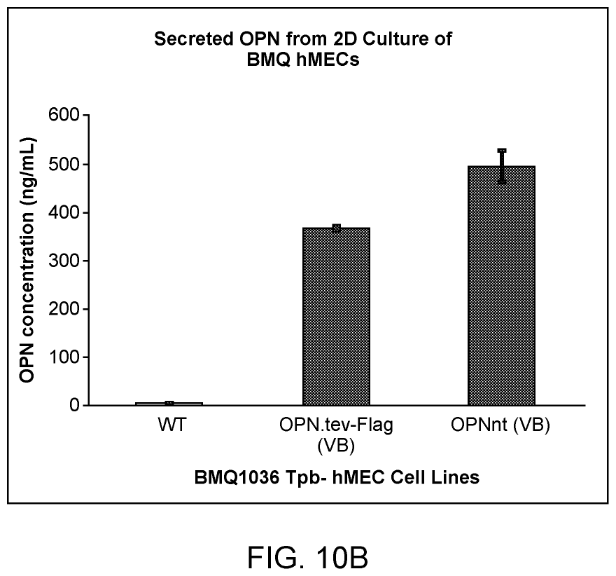

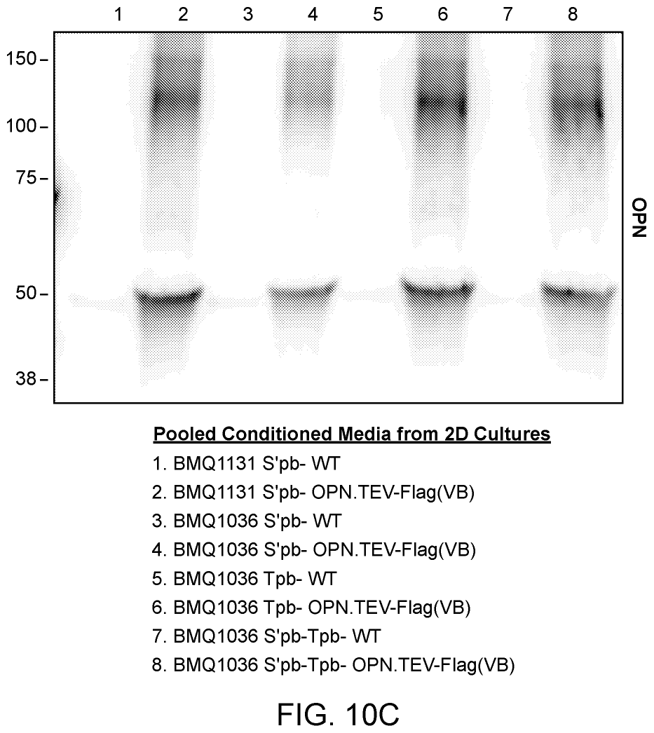

Human mammary epithelial cells (hMEC) isolated and immortalized from donated breast milk were genetically modified to generate cell lines overexpressing secreted, human osteopontin (OPN). Genomic modifications were stably introduced into hMEC using lentiviral vectors, and cell lines with the desired genomic modification were identified via antibiotic selection. Specifically, cells were transduced with MOI of 10 viral particles containing a DNA sequence for the expression of untagged or Flag-tagged OPN protein and antibiotic resistance (FIG. 10A) OPN overexpression was achieved by introduction of an OPN overexpression cassette composed of OPN cDNA driven by a EF1a/CMV promoter into immortalized cells. A first osteopontin construct comprised a secretory signal peptide added to the N-terminus of the OPN protein. A second osteopontin construct comprised FLAG or a 6-Histidine tag (SEQ ID NO: 7) added to the C-terminus of the OPN protein

hMEC were cultured in 2D adherent format for 7 days and conditioned media was analyzed via human OPN ELISA (FIG. 10B). Western blot analysis of OPN was performed from pooled conditioned media across 3 replicates cultured in 2D format for 7 days. Immortalized cell lines over-expressing OPN were compared to immortalized cell lines expressing endogenous OPN levels as a control (FIG. 10C).

The data demonstrate that immortalized hMEC lines transduced with lentiviral vector overexpress OPN when compared to hMEC lines expressing endogenous levels of OPN.

### Example 4: Expansion and Production of OPN in Hollow Fiber and Stir-Tank Bioreactors

The following example demonstrates that production of OPN is scalable across multiple bioreactor platforms.

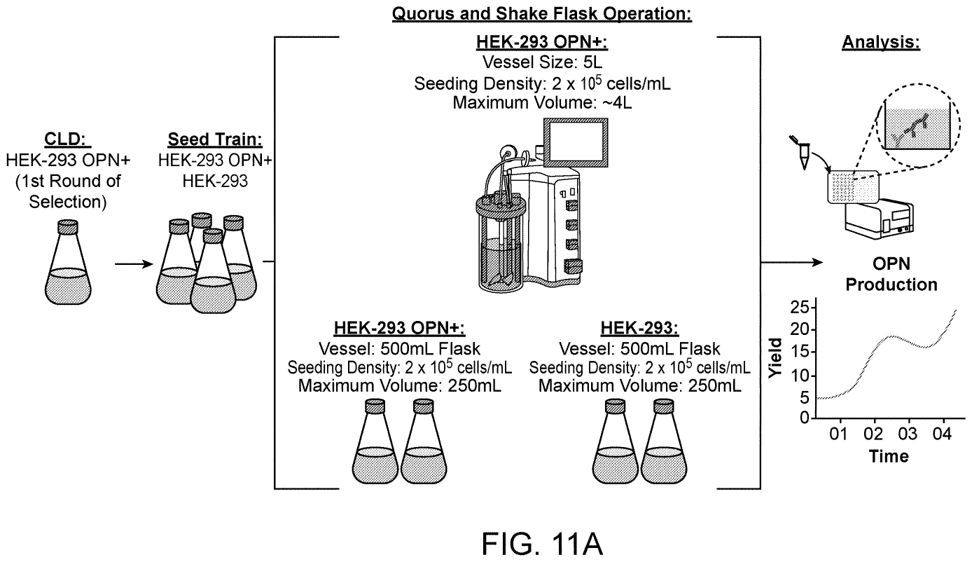

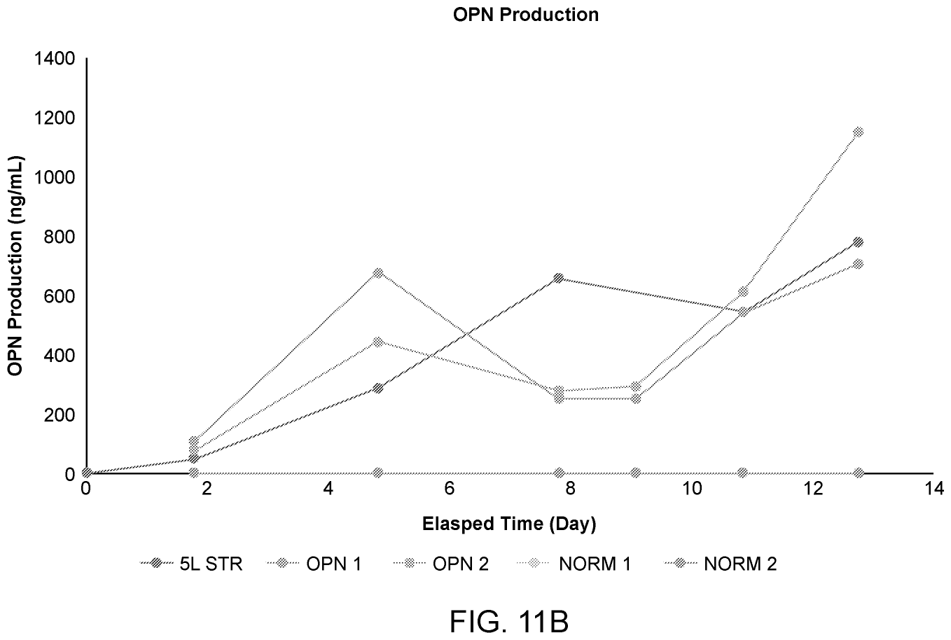

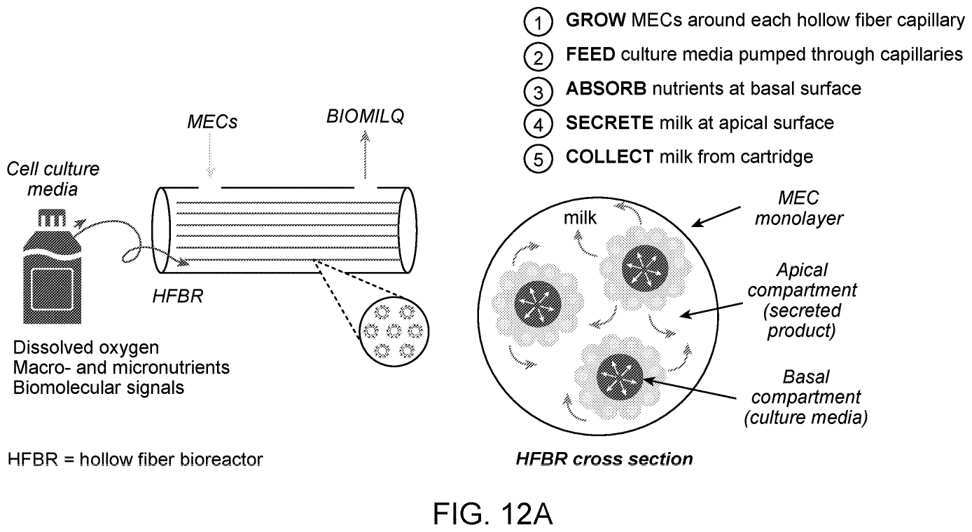

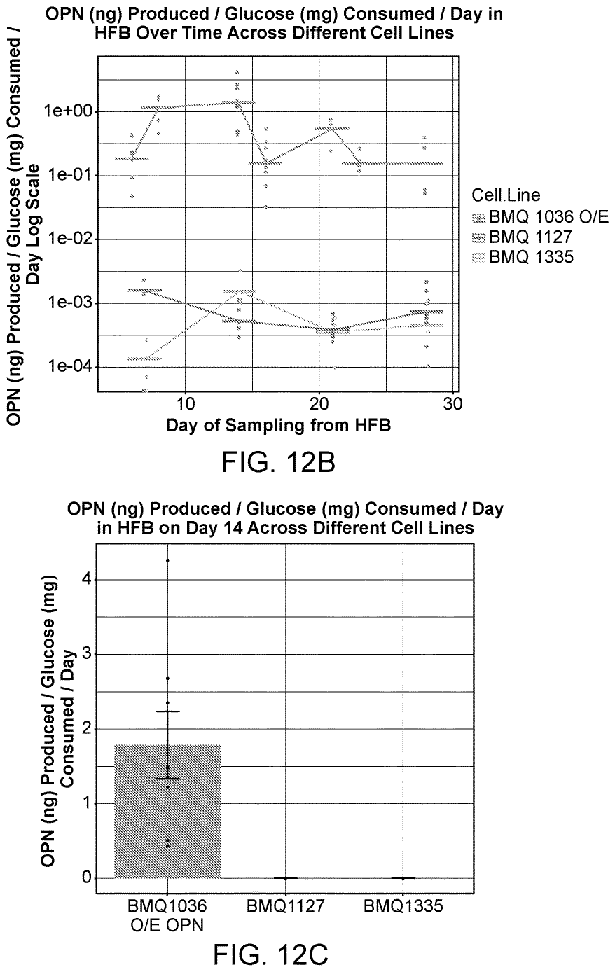

Both hMEC (adherent) and HEK-293 (suspension) cell lines were modified for OPN overexpression, as per the method of Example 3, and tested at bioreactor scale for production. Both hollow fiber bioreactor, (HFBR; hMEC adherent) stir-tank reactor (STR; HEK suspension), and shake flask (HEK suspension) formats were used. OPN production from bioreactor platform runs was then measured by OPN ELISA (FIGS. 11A-11B and 12A-12C).

HEK-293 suspension cell lines were expanded in shakeflasks until 3 different suspension conditions could be seeded at 2×10⁵ cells/mL of culture media: a 5 L stir-tank reactor, a 500 mL shakeflask for wild-type HEK-293 cells (250 mL working volume; NORM1/NORM2), and s 500 mL shakeflask for OPN-overexpressing HEK-293 cells (250 mL volume; OPN1/OPN2). These suspension medias were sampled over a 2 week period and tested for OPN production per mL via OPN ELISA.

Overall OPN production by HEK-293 cell lines was tracked via ELISA per mL of conditioned media. Tracking took place daily for 2 weeks between overexpressing and wild-type cell lines (500 mL shake flasks), as well as at scale (5 L STR using HEK-293 overexpressing cells) (FIGS. 11A-11B).

Multiple immortalized hMEC were expanded and seeded within HFBRs at 40k cells/cm³ and grown for 4 weeks, with media flowing through the intercapillary space. One cell line (BMQ 1036) was genetically modified to overexpress OPN. Extracapillary space was flushed and product collected every 4 days. OPN concentration was measured via ELISA, and the log of average OPN concentration per glucose consumed was plotted over time (FIGS. 12A-12C).

The data demonstrate that in both cases, OPN overexpression was present at larger scale for modified HEK-293 and hMEC cell lines. That is, HEK-293 cells overexpressed OPN when cultured in a suspension production platform, and hMEC isolated from breast milk overexpressed OPN when cultured in an adherent hollow fiber bioreactor platform. Forced overexpression of OPN lead to increased expression in hollow fiber bioreactor product.

### Example 5: Characterization of OPN Phosphorylation Sites and Activity

5.1 Phosphorylation Patterns of OPN from Breast Milk and OPN from HFBR Culture.

The following describes the difference in phosphorylation patterns between OPN obtained from breast milk and OPN obtained from conditioned media produced from HFBR culture.

Breast milk OPN samples were pooled from 10 donors. HFBR OPN samples were obtained by pooling the conditioned media product from an HFBR production run using BMQ 1127 and BMQ 1036 immortalized hMEC lines expressing endogenous OPN. Phosphorylation sites were determined for both breast milk and HFBR OPN samples via untargeted proteomics on phospho-enriched peptides. Breast milk proteins were precipitated using chloroform/methanol followed by solubilization in 5% SDS and detergent-compatible Bradford assay. HFBR product was concentrated using an amicon ultra-4 concentrator. Protein was digested with an S-trap using trypsin, enriched for phosphopeptides using TiO₂, lyophilized, and reconstituted in LC-MS buffer. 2 μL was analyzed by nanoflow LC-DDA-MS/MS. (FIGS. 13A-13C)

The data show that 35 out of 36 known OPN phosphorylation sites were detected in pooled breast milk sample using untargeted phosphoproteomics. Of the 35 known sites detected, 22 were also observed in endogenous OPN produced from HFBRs across two different hMEC cell lines, indicating that endogenous hMEC OPN reproduces may of the phosphorylation sites observed in breast milk OPN.

5.2 Phosphorylation Patterns of OPN from Breast Milk, Commercially Available OPN from HEK293 and Bacterial Cell Culture, and OPN from HFBR Culture.

The following describes the difference in phosphorylation patterns between OPN obtained from breast milk and commercially available human OPN obtained, isolated, and processed via production in HEK293 cells or bacterial cells. The following example also describes the similarity between OPN obtained from breast milk and OPN obtained from conditioned media produced from HFBR culture.

Breast milk OPN sample was obtained from a donor. HFBR OPN sample was obtained from OPN secreted in conditioned media by OPN-expressing cells isolated from said donor's breast milk and grown in HFBR for two weeks. Commercially available human OPN samples that are isolated and lyophilized were obtained from HEK293 cell and bacterial cell production. Phosphorylation sites were determined for donor breast milk OPN, HFBR OPN, and commercially available human OPN samples via targeted proteomics on phospho-enriched peptides. Breast milk proteins were precipitated using chloroform/methanol followed by solubilization in 5% SDS and detergent-compatible Bradford assay. HFBR product was concentrated using an amicon ultra-4 concentrator. Protein was digested with an S-trap using trypsin, enriched for phosphopeptides using TiO₂, lyophilized, and reconstituted in LC-MS buffer. 2 μL was analyzed by nanoflow LC-DDA-MS/MS.

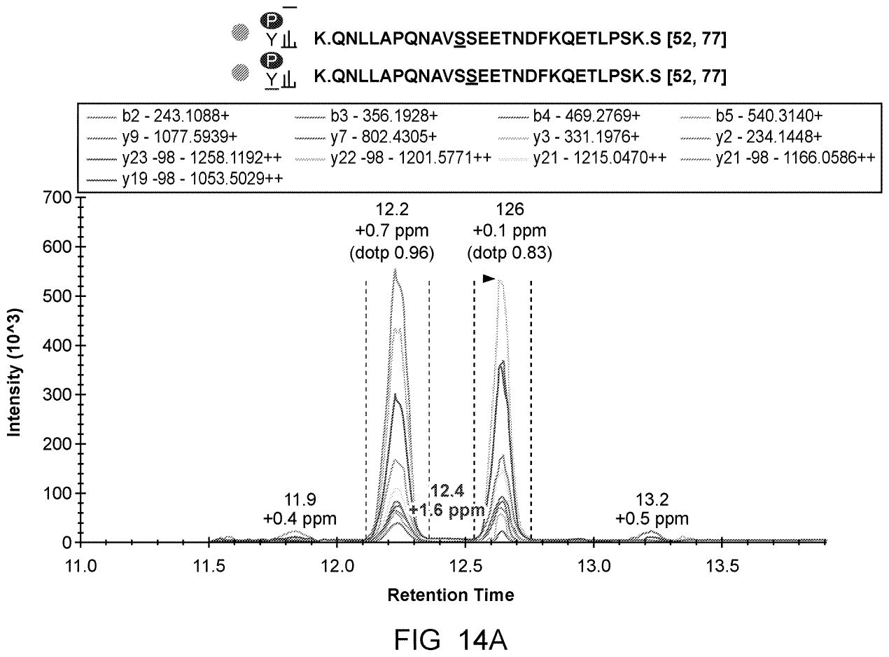

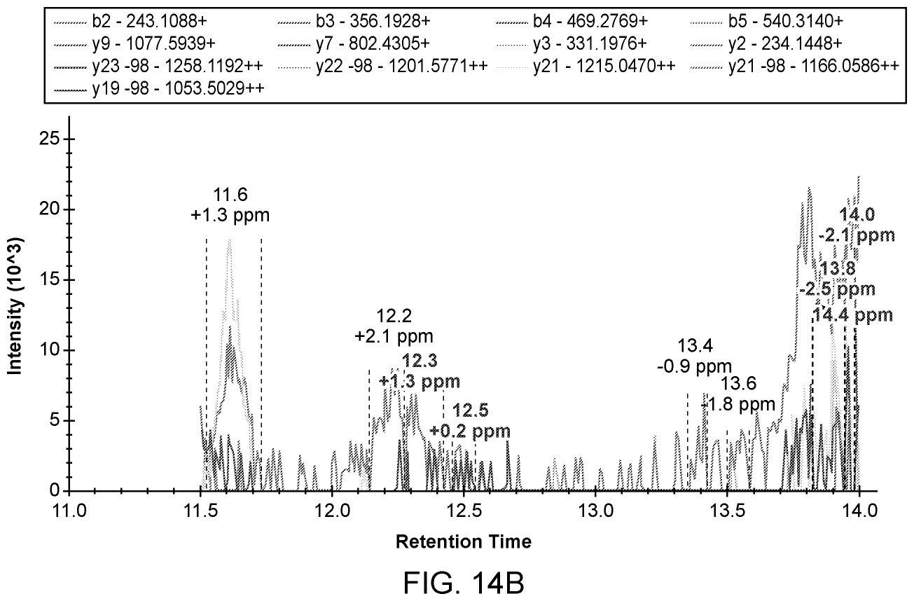

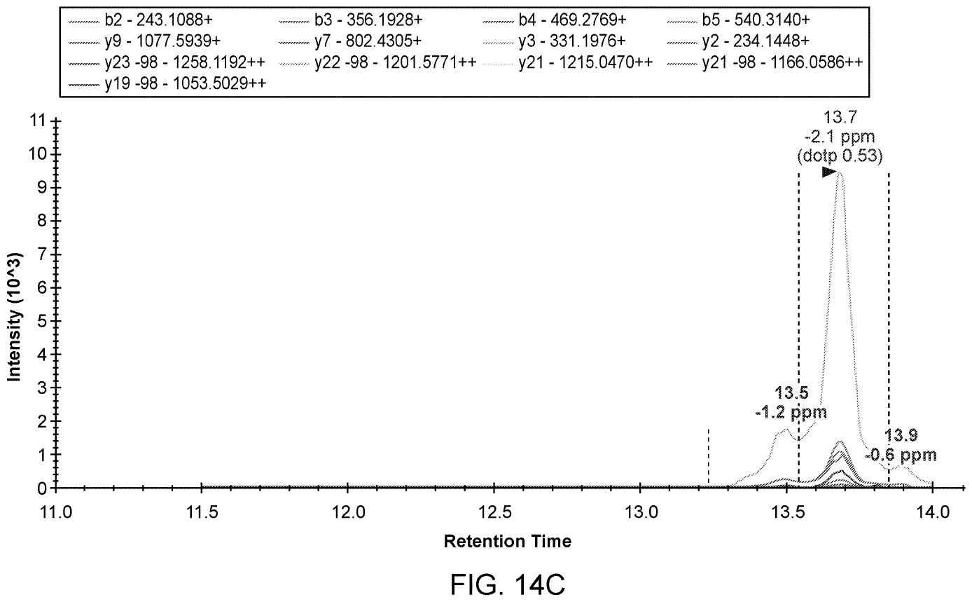

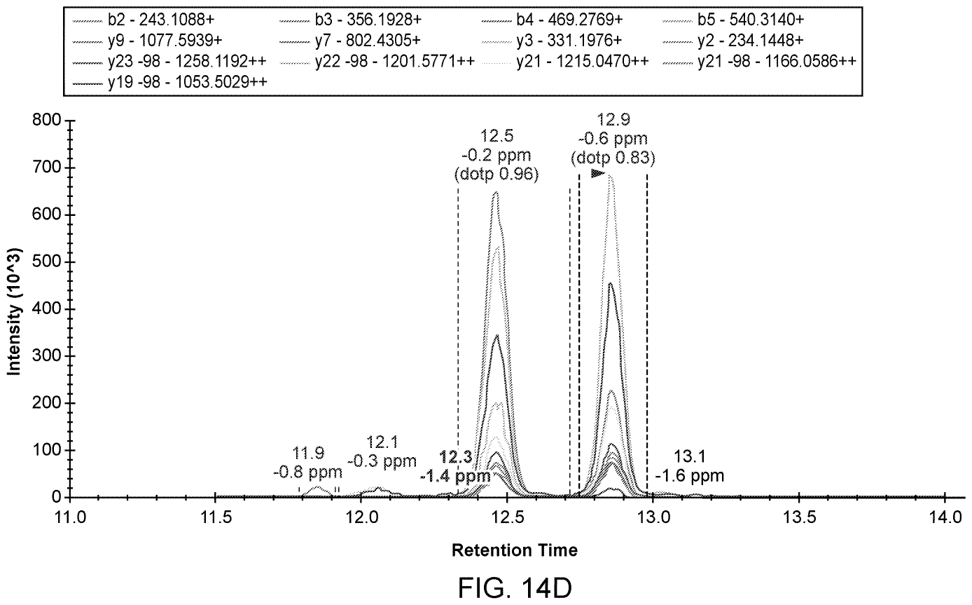

Data is shown for one representative phospho-enriched phosphopeptide of OPN with two isomers from donor breast milk sample (FIG. 14A), commercially available and isolated human samples (FIGS. 14B-14C), and HFBR conditioned media product sample (FIG. 14D). The data show that the conditioned media containing the OPN product secreted by cells from HFBR system reproduced two of the phosphorylation sites observed in human donor breast milk OPN.

### Example 6: HEK-293 Generated OPN Shows Adhesion Activity

The following example shows adhesion activity of non-isolated HEK-293 OPN relative to purified and lyophilized commercially available OPN produced by HEK-293 cells.

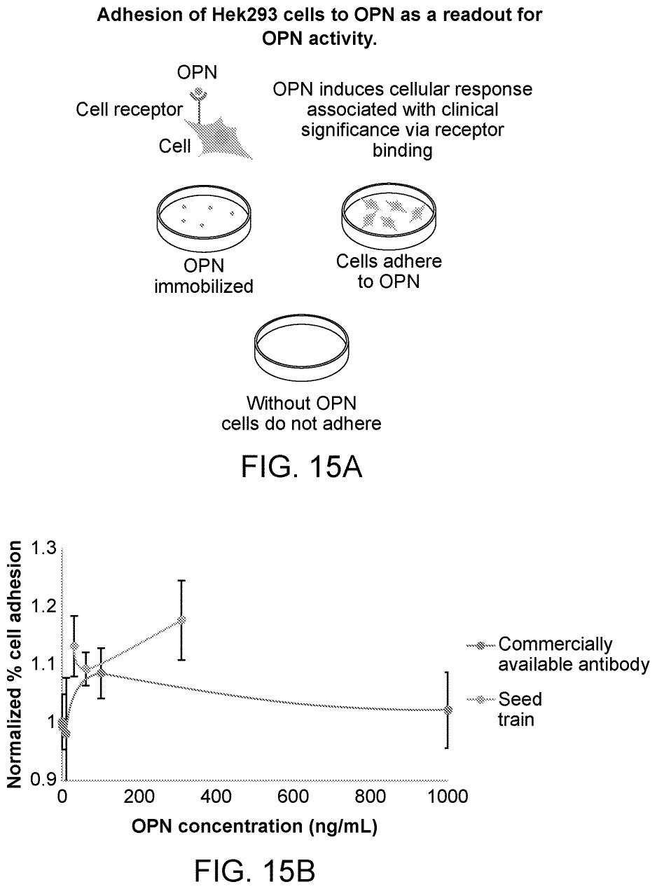

Conditioned media from HEK-293 suspension cells over-expressing OPN and commercially available OPN (n=3) were immobilized. Adhesion of fluorescently adherent HEK-293 cells was measured. Data was represented as average±S.D. % cell adhesion normalized to 0 ng/mL OPN concentration (of commercially obtained OPN). (FIGS. 15A-15B).

The data show that conditioned media containing OPN generated by HEK-293 cells promotes higher cell adhesion at lower concentration compared to isolated commercially available recombinant human OPN

### Example 7: Suspension Adaption of hMEC Cells for Increased Production

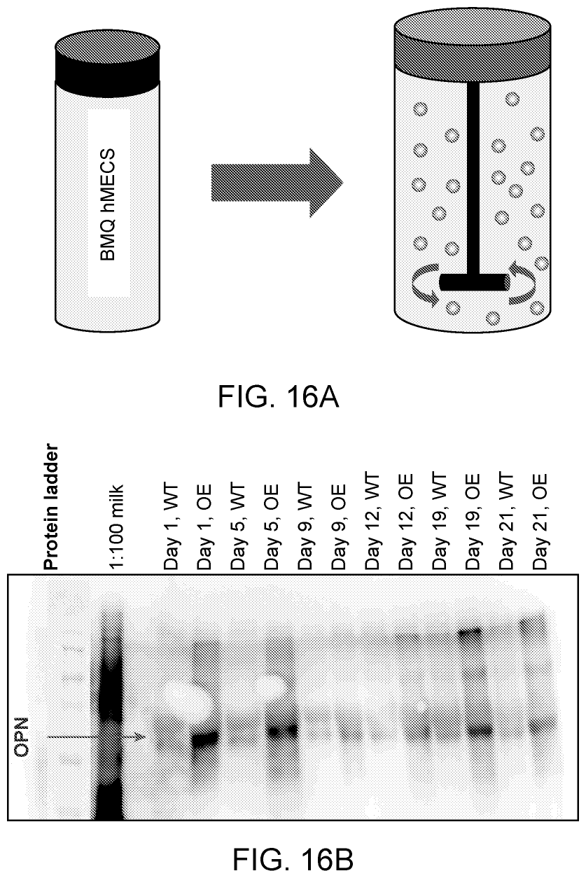

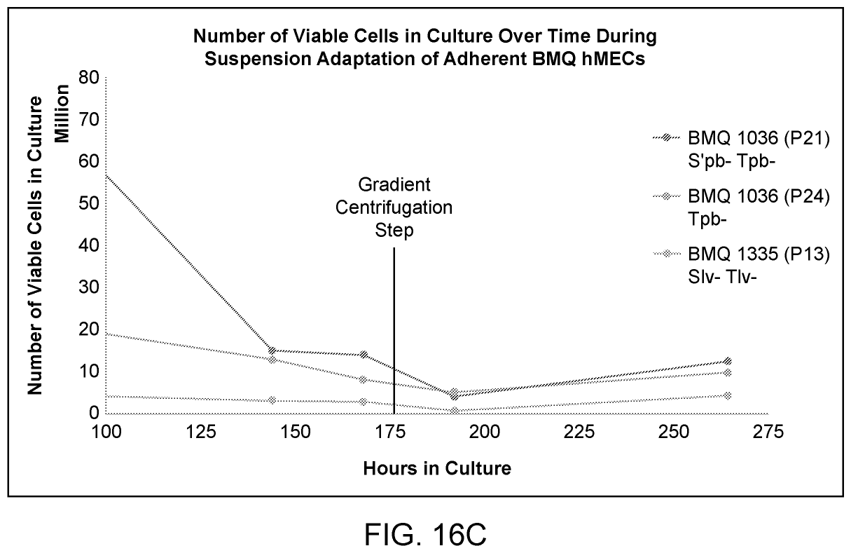

The following example demonstrates the suspension adaptation of adherent hMEC. hMEC lines were cultured in M87A media in non-adherent spinner flasks for 270 hours and the number of viable cells in culture was determined over time to determine the ability of cells to adapt to suspension (FIGS. 16A-16C).

The data show that hMEC retain the ability to overexpress OPN throughout the adaptation process. The adaptation process of adherent hMECs resulted in low viability cultures. The observed increase in viable cell numbers at the end of the adaptation process indicates successful adaptation. Cell populations banked after 270 hours were banked, thawed and expanded successfully (data not shown).

The foregoing examples are illustrative of the present disclosure and are not to be construed as limiting thereof. Although the disclosure has been described in detail with reference to preferred embodiments, variations and modifications exist within the scope and spirit of the disclosure as described and defined in the following claims.

What is claimed is:

1. A recombinant osteopontin comprising one or more post translational modification sites at positions 33, 36, 42, 66, 73, 237, 258, 267, 270, 291, 308, or 311 of SEQ ID NO: 1 or SEQ ID NO: 2.

2. The recombinant osteopontin of claim 1, wherein the post-translational modification is selected from the group consisting of ubiquitination, acylation, isoprenylation, prenylation, alkylation, glycosylation, phosphorylation, and a disulfide bridge.

3. The recombinant osteopontin of claim 1, wherein the recombinant osteopontin comprises post translational modification sites at positions 33, 36, 42, 66, 73, 237, 258, 267, 270, 291, 308, and 311.

4. The recombinant osteopontin of any one of claims 1-2, wherein the recombinant osteopontin is phosphorylated at one or more post translational modification sites at positions 33, 36, 42, 66, 73, 237, 258, 267, 270, 291, 308, or 311 of SEQ ID NO: 1 or SEQ ID NO: 2.

5. The recombinant osteopontin of any one of claims 1-2, wherein the recombinant osteopontin is glycosylated at one or more post translational modification sites at positions 33, 36, 42, 66, 73, 237, 258, 267, 270, 291, 308, or 311 of SEQ ID NO: 1 or SEQ ID NO: 2.

6. The recombinant osteopontin of any one of claims 1-4, wherein the recombinant osteopontin is about 30-75 kilodaltons (kDa) in size.

7. A method of producing recombinant osteopontin from mammary cells, the method comprising:
(a) culturing a live cell construct in a bioreactor under conditions which produce a cultured milk product, said live cell construct comprising:
(i) a three-dimensional scaffold having an exterior surface, an interior surface defining an interior cavity/basal chamber, and a plurality of pores extending from the interior surface to the exterior surface;
(ii) a matrix material disposed on the exterior surface of the three-dimensional scaffold;
(iii) a culture media disposed within the interior cavity/basal chamber and in fluidic contact with the internal surface; and
(iv) an at least 70% confluent monolayer of polarized mammary cells disposed on the matrix material, wherein the mammary cells are modified to overexpress the recombinant osteopontin; and
(b) isolating the recombinant osteopontin from the cultured milk product.

8. The method of claim 7, wherein the mammary cells are modified to overexpress the recombinant osteopontin by expressing a vector comprising a polynucleotide having a sequence as set forth in SEQ ID NO: 3.

9. The method of any one of claims 7-(b), wherein the mammary cells are selected from the group consisting of: primary mammary epithelial cells, mammary myoepithelial cells, mammary progenitor cells, immortalized mammary epithelial cells, immortalized mammary myoepithelial cells, and immortalized mammary progenitor cells.

10. The method of any one of claims 7-9, wherein the mammary cells are immortalized mammary epithelial cells.

11. The method of any one of claims 7-10, wherein the mammary cell is human.

12. The method of any one of claims 7-11, wherein the polarized mammary cells comprise an apical surface and a basal surface.

13. The method of claim 12, wherein the basal surface of the mammary cells is in fluidic contact with the culture media.

14. The method of any one of claims 7-13, wherein the bioreactor is an enclosed bioreactor.

15. The method of any one of claims 7-14, wherein the bioreactor comprises an apical compartment that is substantially isolated from the internal cavity/basal chamber of the live cell construct.

16. The method of any one of claims 7-15, wherein the apical compartment is in fluidic contact with the apical surface of the mammary cells.

17. The method of any one of claims 7-16, wherein the cultured milk product is secreted from the apical surface of the mammary cells into the apical compartment.

18. The method of any one of claims 7-17, wherein the culture media substantially does not contact the cultured milk product.

19. The method of any one of claims 7-18, wherein total cell density of mammary cells within the bioreactor is at least 10¹¹.

20. The method of any one of claims 7-19, wherein total surface area of mammary cells within the bioreactor is at least 1.5 m².

21. The method of any one of claims 7-20, wherein the culture medium comprises a carbon source, a chemical buffering system, one or more essential amino acids, one or more vitamins and/or cofactors, and one or more inorganic salts.

22. The method of any one of claims 7-21, wherein the matrix material comprises one or more extracellular matrix proteins.

23. The method of any one of claims 7-22, wherein the scaffold comprises a natural polymer, a biocompatible synthetic polymer, a synthetic peptide, a composite derived from any of the preceding, or any combination thereof.

24. The method of claim 23, wherein the natural polymer is collagen, chitosan, cellulose, agarose, alginate, gelatin, elastin, heparan sulfate, chondroitin sulfate, keratan sulfate, and/or hyaluronic acid.

25. The method of claim 23, wherein the biocompatible synthetic polymer is polysulfone, polyvinylidene fluoride, polyethylene co-vinyl acetate, polyvinyl alcohol, sodium polyacrylate, an acrylate polymer, and/or polyethylene glycol.

26. The method of any one of claims 7-25, wherein the culturing is carried out at a temperature of about 27 °C to about 39 °C.

27. The method of claim 26, wherein the culturing is carried out at a temperature of about 30 °C to about 37 °C.

28. The method of any one of claims 7-27, wherein the culturing is carried out at an atmospheric concentration of CO2 of about 4% to about 6%.

29. The method of any one of claims 7-28, wherein the culturing is carried out at an atmospheric concentration of CO2 of about 5%.

30. A mammary cell expressing a sequence as set forth in SEQ ID NO: 3.

31. The mammary cell of claim 30, wherein the mammary cell is selected from the group consisting of: primary mammary epithelial cell, mammary myoepithelial cell, mammary progenitor cell, immortalized mammary epithelial cell, immortalized mammary myoepithelial cell, and immortalized mammary progenitor cell.

32. The mammary cell of any one of claims 30-31, wherein the mammary cells is an immortalized mammary epithelial cell.

33. The mammary cell of any one of claims 30-32, wherein the mammary cell is human.
